(19) United States
(12) Patent Application Publication
(10) Pub. No.: US 2023/0233652 A1
(43) Pub. Date: Jul. 27, 2023

(54) 28 KDA GST PROTEINS FROM SCHISTOSOMA FOR THE TREATMENT OF VASCULITIS

(71) Applicant: PAR'IMMUNE SAS, Loos-Lez-Lille (FR)

(72) Inventors: Monique CAPRON, Bougival (FR); Abderrahim LACHGAR, Paris (FR)

(73) Assignee: PAR'IMMUNE SAS, Loos-Lez-Lille (FR)

(21) Appl. No.: 17/919,002

(22) PCT Filed: Apr. 16, 2021
(86) PCT No.: PCT/EP2021/060001

§ 371 Date: Oct. 14, 2022

(30) Foreign Application Priority Data
Apr. 16, 2020 (EP) 20169888.3

(51) Int. Cl. A61K 38/45; A61P 37/06; A61P 9/14

(52) U.S. Cl. CPC A61K 38/45; A61P 37/06; A61P 9/14

(57) ABSTRACT

Polypeptides that are glutathione-S-transferases originating from different schistosome parasites, as well as nucleic acids, vectors, compositions or kits, for use in the preventive or therapeutic treatment of vasculitis or of a disease characterized by a M1/M2 macrophage ratio dysregulation, such as e.g. a decrease of the M1-type immune response and/or an increase of the M2-type immune response.

### FIELD OF INVENTION

The present invention relates to particular polypeptides which are glutathione-S-transferases originating from various schistosome parasites, as well as nucleic acids, vectors, compositions or kits, for use in the preventive or therapeutic treatment of vasculitis or of any disease characterized by a M1/M2 macrophage ratio dysregulation.

### BACKGROUND OF INVENTION

Vasculitis is a group of rare diseases that have in common inflammation of blood vessels. These vessels include arteries and veins. There are many types of vasculitis, and they may vary greatly in symptoms, severity and duration. Most types of vasculitis are rare and the causes are generally not known. Vasculitis affects people of both sexes and all ages.

Symptoms Associated to Vasculitis

The symptoms of vasculitis are particular to the blood vessels that are involved in the inflammatory response. Although the different types of vasculitis have localized patterns of blood vessel involvement, vasculitis is a systemic disease resulting in typical symptoms of inflammation, such as fever, fatigue, weight loss, a rapid pulse, and aches and pains. Virtually every organ system may be affected by vasculitis, including skin, lungs, joints, kidneys, gastrointestinal tract, blood, eyes, brain, nerves, sinuses, nose and throat. The pattern of organ involvement is specific to the individual as well as the type of vasculitis.

According to the International Chapel Hill Consensus Conference on the Nomenclature of Vasculitides (CHCC 2012), vasculitis can be classified according to:
a) the size of vessels involved, which may be:
   large vessels (e.g. aorta, coronary arteries)
   medium vessels (e.g. medium or small arteries)
   small vessels (e.g. Antineutrophil cytoplasmic antibody (ANCA)-associated vasculitis, immune complex)
   variable vessels (Behçet's disease, Cogan disease)
b) the organs or tissues involved, which may be:
   multi-organs (Behçet's disease, immune complex)
   single organs (skin, testicular, central nervous system . . . )
c) association with other diseases (e.g. lupus, rheumatoid arthritis, syphilis, cancer . . . ).

Behçet's Disease

Behçet's disease (BD) is a chronic, relapsing, multiorgan autoimmune inflammatory disease of unknown origin. Pathologically, the disease is characterized by systemic necrotizing vasculitis of small and large vessels, and arthritis. Oral ulcers, genital ulcers, cutaneous lesions, ocular (uveitis) and articular involvement are the most frequent features of the disease. The involvement of the gastrointestinal tract, central nervous system and large vessels is less frequent (Baharav et al. 2006 Drug Discovery Today: Disease Models 3(1):11-14). Mucocutaneous lesions are considered hallmarks of the disease, and often precede other manifestations (Alpsoy 2016 Journal of Dermatology 43(6):620-632).

Behçet's disease usually starts around the third or fourth decade of life. Sex distribution is roughly equal. The diagnosis is based on clinical criteria, as there is no pathognomonic test (Alpsoy 2016 Journal of Dermatology 43(6):620-632).

Behçet's disease is mainly distributed along the ancient Silk Road from Mediterranean countries, including Turkey (370 cases per 100,000 population), to Middle Eastern and East Asian countries, but Behçet's disease is rarely encountered in Northern Europe (0.64 cases per 100,000 population), North America (0.12-0.33 cases per 100,000 population), Australia, and Africa (Tong et al. 2019 Front. Immunol. 10(665)).

Regarding clinical manifestations, oral aphthosis is seen in more than 95% of patients, genital aphthosis in 60-90% of patients, skin manifestations (pseudofolliculitis, erythema nodosum) in 40-90% of patients, eyes manifestations (uveitis/retinal vasculitis) in 45-90% of patients, gastrointestinal manifestations (diarrhea, hemorrhage, perforation, pain) in 4-38% of patients, vascular manifestations (venous/arterial thrombosis, aneurysm) in 2.2-50% of patients, neurological manifestations (all kinds, especially meningo-encephalitis) in 2.3-38.5% of patients, and articular manifestations (arthralgia, arthritis, ankylosing spondylitis) in 11.6-93% of patients (Davatchi et al. 2017 Expert Review of Clinical Immunology 13(1):57-65).

Genetic factors such as HLA-B*51, as well as environmental factors, including microbial components, have been implicated in the pathogenesis of Behçet's disease (Nakano et al. 2018 Arthritis Research & Therapy 20:124).

The main hypotheses regarding the pathophysiology include the following: neutrophil hyperactivity, autoimmune reaction to self-antigens (such as heat shock proteins, S-antigen or α-tropomyosin), immune-complex formation, and viral or bacterial infection) (Baharav et al. 2006 Drug Discovery Today: Disease Models 3(1):11-14).

Innate Immune Cells

Innate immune cells are involved in the pathogenesis of Behçet's disease, in particular macrophages, neutrophils, natural killer (NK) cells and γδ T cells (Tong et al. 2019 Front. Immunol. 10(665)).

M1/M2 Macrophages

Macrophages are important cells of the innate immune system that help fighting against pathogens. Macrophages are also crucial in the pathogenesis of immune-inflammatory disorders. Macrophages were initially thought to only promote inflammation, but it was later discovered that they have the ability to both promote as well as resolve inflammation. This inflammation and resolution paradox was solved after the discovery of the two macrophage subsets: M1 and M2. The naïve (M0) macrophages can polarize under different conditions to become either M1 or M2 macrophages.

The M1 macrophages, also known as “classical macrophages”, are pro-inflammatory as they are involved in killing microbes and causing inflammation. M1 macrophage subset is activated by microbial products such as lipopolysaccharide (LPS), or pro-inflammatory cytokines such as interferon-γ. They kill pathogens by releasing reactive oxygen and nitrogen species as well as pro-inflammatory cytokines, including IL-6, IL-12, IL-23, IL-1β, and tumor necrosis factor (TNF)-α.

On the other hand, the M2 macrophages, also known as “alternatively activated macrophages”, have major roles in the resolution of inflammation, angiogenesis, tissue remodeling, and repair. These macrophages are stimulated by cytokines like IL-4, IL-10, or IL-13 and, in turn, produce remodeling factors such as tissue inhibitor of metalloproteinase (TIMP1) and transforming growth factor (TGF-β), chemokines such as macrophage derived chemokine (MDC) and thymus and activation-regulated chemokine (TARC), anti-inflammatory cytokines such as IL-10, or Arginase-1, macrophage colony stimulating factor (M-CSF).

Human adenosine deaminase type 2 deficiency (DADA2), which is due to bi-allelic deleterious mutations in the ADA2 gene, is the first described monogenic type of small- and medium-size vessel vasculitis. In DADA2, monocyte/macrophage differentiation is skewed to a pro-inflammatory M1 subset, which is detrimental for endothelial integrity (Moens et al. 2019 Immunol Rev. 287(1):62-72).

Besides, IL10 and related genes were identified as susceptibility genes associated with immune-mediated diseases including Behçet's disease, suggesting the involvement of abnormal M2 macrophage function in the pathogenesis of this disease. Indeed, dysfunction of M2 macrophages has been shown to exacerbate inflammation in a herpes simplex virus-induced Behçet's disease mouse model. Behçet's disease skin lesions show M1 macrophages predominance compared with systemic sclerosis skin lesions. Single-nucleotide polymorphism may contribute to M1 macrophage-predominant inflammation in Behçet's disease, and the skewed macrophage polarization may be correctable by immunological intervention (Nakano et al. 2018 Arthritis Research & Therapy 20:124).

Neutrophils

Neutrophils in Behçet's disease patients exhibit high intrinsic activation that may be associated with HLA-B*51. Neutrophils are usually involved in perivascular infiltration in Behçet's disease lesions. Indeed, in the acute phase, neutrophils predominate in the vasculitic infiltrate, later replaced by CD4+ T cells plasma cells and macrophages (Baharav et al. 2006 Drug Discovery Today: Disease Models 3(1):11-14). The production of reactive oxygens species (ROS) is a normal characteristic of neutrophils. Neutrophil-mediated oxidative stress abnormalities may play an important role in the pathogenesis of Behçet's disease, and advanced oxidation protein products (AOPPs) may be a useful marker for monitoring the progression and severity of disease activity in patients with Behçet's disease. Histopathological analysis has shown that arteries and veins are infiltrated by neutrophils and lymphocytes, which results in vascular endothelial dysfunction. Endothelial dysfunction and neutrophil vascular inflammation are key factors mediating thrombosis in patients with Behçet's disease (Tong et al. 2019 Front. Immunol. 10(665)).

NK Cells

NK cells not only play a cytotoxic role in infected cells and tumor cells but also regulate the function of other immune cells by secreting cytokines. The number of NK cells in the peripheral blood of Behçet's disease patients is significantly decreased. Peripheral blood depletion of NK cells in Behçet's disease patients may reflect increased homing of these cytotoxic cells to inflammatory sites. It was reported an advantage of the NK1 subset in Behçet's disease patients and, compared with healthy subjects, the proportions of NK2 and IL-10-secreting cells in Behçet's disease patients were lower. IL-10 has especially important anti-inflammatory and immunosuppressive effects. Because of the inhibitory effect of IFN-γ, the dominant function of NK1 cells was increased, and increased secretion of IFN-γ may inhibit NK2 cells in Behçet's disease patients. NK cells may play an active role in the remission of Behçet's disease patients through NK2 polarization (Tong et al. 2019 Front. Immunol. 10(665)).

γδ T Cells

The major subset of γδ T cells in the peripheral blood can produce multiple proinflammatory cytokines in the presence of growth factors and cytokines. In particular, IL-1 and IL-23 have been shown to mediate autoimmune inflammatory diseases. These cytokines activate γδ T cells which are important sources of innate IL-17 and IL-21 production (Tong et al. 2019 Front. Immunol. 10(665)).

Cytokines Produced by Innate Immune Cells

Proinflammatory Cytokines

In Behçet's disease patients, the production of proinflammatory cytokines by innate immune cells is enhanced, such as e.g. IL-1, IL-6, TNF-α, IFN-γ, IL-21, IL-23 and TGF-β (Tong et al. 2019 Front. Immunol. 10(665)).

IL-1, IL-6, and TNF-α are major proinflammatory cytokines in patients with Behçet's disease. These cytokines have been found in the ocular fluid of patients with Behçet's disease for more than 20 years and are believed to be the major inflammatory mediators leading to the development of the disease.

IL-6 is clearly a pleiotropic cytokine, which is produced by innate immune cells. IL-6 production is tightly negatively regulated, and abnormal excessive production of IL-6 has been found to be related to autoimmune and chronic inflammatory diseases. The increase in IL-6 in the CSF of patients with neuro-Behçet's disease was reported to be associated with long-term prognosis and disease activity and is regarded as a marker of disease activity.

TNF-α is a representative proinflammatory cytokine and plays a central role in the induction and maintenance of inflammation in the autoimmune response. In inflammatory diseases, TNF-α is mainly produced by cells of the monocyte/macrophage lineage. Over the past decade, the off-label use of TNF-α antagonists such as infliximab, adalimumab, etanercept and golimumab has improved the treatment of refractory immune-mediated uveitis, especially in Behçet's disease, and there is sufficient evidence to suggest that TNF-α inhibition is an important development in the treatment of patients with severe and resistant Behçet's disease (Tong et al. 2019 Front. Immunol. 10(665)).

Anti-Inflammatory Cytokines

Conversely, the level of some anti-inflammatory cytokines is low in Behçet's disease patients. IL-37 was first described as an anti-inflammatory cytokine in autoimmune and inflammatory diseases. In the serum and PBMC culture supernatants from patients with active Behçet's disease, the level of IL-37 was reported to be decreased (Tong et al. 2019 Front. Immunol. 10(665)).

Besides, expression studies have shown that disease-associated IL-10 variants are associated with reduced expression of this anti-inflammatory cytokine, which may lead to a susceptible inflammatory state, thus increasing susceptibility to Behçet's disease (Tong et al. 2019 Front. Immunol. 10(665)).

Therapeutic Approaches

The main treatment used worldwide in patients with Behçet's Disease is Colchicine.

In parallel, in some regions, clinicians use immunosuppressors in patients with severe disease. Some treatments for Behçet's disease target the innate immune response. These include TNF-α antagonists (mainly used in Japan and Israel) and IFN-α, as well as the use of agents that target interleukins and their receptors, such as IL-1 blockers (Anakinra and Canakinumab), IL-6 blocker (Tocilizumab), and a monoclonal antibody targeting IL-12/IL-23 (Ustekinumab) (Tong et al. 2019 Front. Immunol. 10(665)).

A treatment strategy could also be directed at enhancement of IL-10 production or local accumulation of M2 macrophages into inflammatory lesions, contributing to the improvement of clinical outcomes of Behçet's disease. TNF inhibitors may lead to a relative enrichment of M2 macrophages by inhibiting M1 macrophage function.

Alternatively, Apremilast, a phosphodiesterase 4-selective inhibitor for which an international clinical trial for Behçet's disease is ongoing, stimulates the production of IL-10 and downregulates that of proinflammatory cytokines. Clinical efficacy of these treatments is associated with corrected M1/M2 balance (Nakano et al. 2018 Arthritis Research & Therapy 20:124). Recently, the FDA approved the use of Apremilast administered at 30 mg twice daily for the treatment of adults with oral and skin ulcers associated with Behçet's Disease.

Although treatment has become much more effective in recent years with the introduction of newer drugs, Behçet's disease is still associated with considerable morbidity and increased mortality.

Therefore, there is still a real and urgent need to develop a novel preventive and/or therapeutic treatment for vasculitis in general and for Behçet's disease.

Moreover, different patients with Behçet's disease may experience different symptoms affecting different organs. Clinicians often prescribe different products to patients, depending on the affected organs, in order to treat the particular symptoms they experience. For instance, according to the main recommendations:
patients having skin, mucosal or joint lesions are given topical corticosteroids and Colchicine, Lactobacilli lozenges, Azathioprine, IFN-α and Etanercept;
patients having uveitis and venous thrombosis are given Azathioprine, IFN-α, Infliximab or Adalimumab;
patients having pulmonary aneurysms or peripheral aneurysms undergo surgery and receive Cyclophosphamide and Infliximab;
patients having CNS involvement or gastrointestinal involvement are given topical and/or oral 5-ASA derivatives and Azathioprine, Infliximab or Adalimumab.

Therefore, there is an urgent need to develop a novel product capable of globally preventing and/or treating the multi-organ lesions observed in systemic inflammatory diseases like vasculitis and Behçet's disease.

Animal Models of BehçEt's Disease

Concerning ANCA-Associated Vasculitis (AAV), to date there is no good model that replicate the granulomatous lesions found in granulomatosis with polyangiitis (GPA, formerly Wegener's), or the development of vasculitis lesions in organs other than the lungs or kidneys. However, use of a combination of the available models should allow greater understanding of the critical requirements for disease.

There is a relatively low number of animal models suitable to investigate the pathogenesis of Behçet's Disease (BD). This is often the case for autoimmune and autoinflammatory diseases, which involve a combination of genetic and environmental factors, leading to dysregulation of immune system. To test and understand immunopathogenesis of Behçet's disease, animal models were developed based on environmental pollutants, bacterial and human heat shock protein derived peptides, and virus injections. Using these animal models separately and/or concurrently allows for a more effective investigation into Behçet's disease.

The group of Sohn et al., in Korea, developed a Herpes Simplex Virus (HSV)-induced model in ICR mice (“BD mice”) which produced Behçet's disease-like symptoms similar to those observed in patients, including oral, genital, and skin ulcers, eye lesions, arthritis, and intestinal involvement (Sohn et al. 2012 Clin Exp Rheumatol 30(Suppl.72): S96-S103). However, whereas this model is well managed in Korea it appeared difficult and long to set up outside Korea.

The team of Stanford et al, in UK, developed a model based on the use of HSP (Heat Shock Protein) in Lewis rats (Stanford et al 1994 Clin Exp Immunol 97:226-31). However, this model is restricted to uveitis symptoms and it is thus not fully satisfying as a model of Behçet's disease.

The group of Mor et al, in Israel, used α-tropomyosin as a target self-antigen to induce anterior uveitis, joint and skin lesions, after injection to Lewis rats in the presence of Complete Freund adjuvant (Mor et al 2002 Eur. J. Immunol. 32:356-365). Tropomyosin is a self-antigen present in numerous tissues and recognized by sera from BD patients. Induction of an autoimmune pathogenicity was shown after immunization of rats with α-tropomyosin in the presence of Complete Freund's Adjuvant since rats develop anterior uveitis and skin inflammation (Baharav et al. 2006 Drug Discovery Today: Disease Models 3(1):11-14). The cytokine profile of pathogenic cells had a M1/Th1 pattern.

In conclusion, no ideal model of Behçet's disease faithfully reproducing all the aspects of the human disease is available today. However, current animal models provided useful information regarding the pathophysiology of BD. In particular, these models are useful to relate the cellular and cytokine modifications to the immunopathology of BD.

The inventors have used the imiquimod mouse model and the α-tropomyosin/Lewis rat model to evaluate the effect of P28GST immunization on the clinical symptoms (eyes, joints and skin) and on the regulation of immune response. Indeed, if the α-tropomyosin/Lewis rat model induces an autoimmune pathogenicity mimicking three Behçet's disease symptoms, the imiquimod mouse model, classically used as an animal model of skin inflammation, can also be employed to develop potency assay of new molecules in the treatment of Behçet's disease. Indeed, the imiquimod model induces very similar immune disorders to those observed in Behçet's disease patients such as:
increase of TNF-α, IFN-γ, IL-23 and IL-6 cytokines,
neutrophil involvement,
increase of VEGF (induction of strong angiogenesis and vasculitis),
M2 macrophages involvement to improve these two immune-mediated inflammatory diseases.

Inventors' Surprising Discovery

Parasites induce an immune response in the infected host. In particular, helminth parasites, such as schistosomes, are potent regulators of the host immune system.

The inventors have shown that P28GST proteins from Schistosoma are capable of inducing an anti-inflammatory immune response (notably mediated by M2 macrophages) and/or reducing or suppressing the inflammatory immune response (notably mediated by M1 macrophages). Indeed, the inventors have surprisingly shown that P28GST proteins from Schistosoma are capable of inducing M2 macrophages and/or reducing the M1-type macrophage immune response (including e.g, the number of M1 macrophages and the level of M1-associated molecules). This decrease in the M1-type response allows reducing the symptoms associated with inflammation observed in vasculitis. In particular, the inventors have surprisingly shown that the P28GST proteins decrease the secretion of pro-inflammatory cytokines and/or mediators known to be produced notably by M1 macrophages and/or increase the secretion of anti-inflammatory cytokines and/or mediators known to be produced notably by M2 macrophages. These molecules circulate throughout the body and their decrease or their increase potentially affects all organs.

Therefore, the P28GST proteins can therefore be used for decreasing the M1-type immune response and/or increasing the M2-type immune response, in a subject in need thereof.

Various diseases have been shown to be associated to a M1/M2 macrophage ratio dysregulation, as such e.g. atherosclerosis, endometriosis, hypertension, osteonecrosis, Parkinson's disease, steatohepatitis, obesity-induced pathologies, lipodystrophy and myocardial infarction.

Thus, the P28GST proteins are also useful in the preventive or therapeutic treatment of diseases characterized by a M1/M2 macrophage ratio dysregulation.

Finally, since they act on the cytokines and/or mediators that are present throughout the body, the P28GST proteins can be used for the preventive or therapeutic treatment of multi-organ diseases affecting any organ or even the whole body, such as vasculitis.

### SUMMARY

The invention relates to a polypeptide comprising, or consisting of, an amino acid sequence selected from the group consisting of:
a) the sequence of SEQ ID NO: 1, SEQ ID NO: 2, SEQ ID NO: 3, SEQ ID NO: 5 SEQ ID NO: 6, SEQ ID NO: 7 or SEQ ID NO: 8;
b) a fragment of a sequence defined in a), provided that said polypeptide decreases the M1-type immune response and/or increases the M2-type immune response; and
c) a sequence having at least 80% of identity with a sequence defined in a) or b), provided that said polypeptide decreases the M1-type immune response and/or increases the M2-type immune response;

for decreasing the M1-type immune response and/or increasing the M2-type immune response, in a subject in need thereof.

According to an embodiment, said polypeptide is for use in the preventive or therapeutic treatment of:
vasculitis, or
a disease characterized by a M1/M2 macrophage ratio dysregulation selected from the group consisting of atherosclerosis, endometriosis, hypertension, osteonecrosis, Parkinson's disease, steatohepatitis, obesity-induced pathologies, lipodystrophy and myocardial infarction.

This invention relates to a polypeptide comprising, or consisting of, an amino acid sequence selected from the group consisting of:
a) the sequence of SEQ ID NO: 1, SEQ ID NO: 2, SEQ ID NO: 3, SEQ ID NO: 5 SEQ ID NO: 6, SEQ ID NO: 7 or SEQ ID NO: 8;
b) a fragment of a sequence defined in a), provided that said polypeptide decreases the M1-type immune response and/or increases the M2-type immune response; and
c) a sequence having at least 80% of identity with a sequence defined in a) or b), provided that said polypeptide decreases the M1-type immune response and/or increases the M2-type immune response;
for use in the preventive or therapeutic treatment of vasculitis.

According to an embodiment, said polypeptide decreases the M1-type immune response and/or increases the M2-type immune response.

According to an embodiment, said fragment comprises:
a fragment having the amino acid sequence ranging from the amino acid at position 24 to the amino acid at position 43 of SEQ ID NO: 1 (SEQ ID NO: 19), of SEQ ID NO: 2 (SEQ ID NO: 20), of SEQ ID NO: 3 (SEQ ID NO: 21) or of SEQ ID NO: 5 (SEQ ID NO: 22);
a fragment having the amino acid sequence ranging from the amino acid at position 115 to the amino acid at position 131 of SEQ ID NO: 1 (SEQ ID NO: 23), of SEQ ID NO: 2 (SEQ ID NO: 24), of SEQ ID NO: 3 (SEQ ID NO: 25) or of SEQ ID NO: 5 (SEQ ID NO: 26); and/or
a fragment having the amino acid sequence ranging from the amino acid at position 190 to the amino acid at position 211 of SEQ ID NO: 1 (SEQ ID NO: 27), of SEQ ID NO: 2 (SEQ ID NO: 28), of SEQ ID NO: 3 (SEQ ID NO: 29) or of SEQ ID NO: 5 (SEQ ID NO: 30).

According to an embodiment, said fragment comprises:
a fragment having the amino acid sequence ranging from the amino acid at position 15 to the amino acid at position 60 of SEQ ID NO: 1 (SEQ ID NO: 31), of SEQ ID NO: 2 (SEQ ID NO: 32), of SEQ ID NO: 3 (SEQ ID NO: 33) or of SEQ ID NO: 5 (SEQ ID NO: 34);
a fragment having the amino acid sequence ranging from the amino acid at position 100 to the amino acid at position 150 of SEQ ID NO: 1 (SEQ ID NO: 35), of SEQ ID NO: 2 (SEQ ID NO: 36), of SEQ ID NO: 3 (SEQ ID NO: 37) or of SEQ ID NO: 5 (SEQ ID NO: 38); and/or
a fragment having the amino acid sequence ranging from the amino acid at position 170 to the amino acid at position 211 of SEQ ID NO: 1 (SEQ ID NO: 39), of SEQ ID NO: 2 (SEQ ID NO: 40), of SEQ ID NO: 3 (SEQ ID NO: 41) or of SEQ ID NO: 5 (SEQ ID NO: 42).

According to another embodiment, said fragment comprises:
a fragment having the amino acid sequence ranging from the amino acid at position 21 to the amino acid at position 43 of SEQ ID NO: 6 (SEQ ID NO: 43), of SEQ ID NO: 7 (SEQ ID NO: 44) or of SEQ ID NO: 8 (SEQ ID NO: 45);
a fragment having the amino acid sequence ranging from the amino acid at position 112 to the amino acid at position 125 of SEQ ID NO: 6 (SEQ ID NO: 46), of SEQ ID NO: 7 (SEQ ID NO: 47) or of SEQ ID NO: 8 (SEQ ID NO: 48); and/or
a fragment having the amino acid sequence ranging from the amino acid at position 181 to the amino acid at position 217 of SEQ ID NO: 6 (SEQ ID NO: 49), or from the amino acid at position 172 to the amino acid at position 187 of SEQ ID NO: 7 (SEQ ID NO: 50), or from the amino acid at position 181 to the amino acid at position 203 of SEQ ID NO: 8 (SEQ ID NO: 51).

Thus, according to an embodiment, said fragment has an amino acid sequence selected from the group consisting of SEQ ID NO: 19 to SEQ ID NO: 51.

According to another embodiment, said polypeptide comprises, or consists of, an amino acid sequence selected from the group consisting of:
a) the sequence of SEQ ID NO: 1;
b) a fragment of a sequence defined in a), provided that said polypeptide decreases the M1-type immune response and/or increases the M2-type immune response; and
c) a sequence having at least 80% of identity with a sequence defined in a) or b), provided that said polypeptide decreases the M1-type immune response and/or increases the M2-type immune response.

According to an embodiment, said fragment comprises:
a fragment having the amino acid sequence ranging from the amino acid at position 24 to the amino acid at position 43 of SEQ ID NO: 1 (SEQ ID NO: 19);
a fragment having the amino acid sequence ranging from the amino acid at position 115 to the amino acid at position 131 of SEQ ID NO: 1 (SEQ ID NO: 23); and
a fragment having the amino acid sequence ranging from the amino acid at position 190 to the amino acid at position 211 of SEQ ID NO: 1 (SEQ ID NO: 27).

According to another embodiment, said polypeptide comprises, or consists of, an amino acid sequence selected from the group consisting of:
a) the sequence of SEQ ID NO: 1;
b) a fragment having an amino acid sequence selected from the group consisting of SEQ ID NO: 19 to SEQ ID NO: 30; and
c) a sequence having at least 80% of identity with a sequence defined in a) or b), provided that said polypeptide decreases the M1-type immune response and/or increases the M2-type immune response.

The invention also relates to a nucleic acid encoding a polypeptide as described herein, or a vector comprising said nucleic acid, for use in the preventive or therapeutic treatment of vasculitis, or of a disease characterized by a M1/M2 macrophage ratio dysregulation selected from the group consisting of atherosclerosis, endometriosis, hypertension, osteonecrosis, Parkinson's disease, steatohepatitis, obesity-induced pathologies, lipodystrophy and myocardial infarction.

The invention also relates to a nucleic acid encoding a polypeptide as described herein, or a vector comprising said nucleic acid, for use in the preventive or therapeutic treatment of vasculitis.

Another object of the invention is a composition comprising a polypeptide as described herein, or a nucleic acid or a vector as described herein, for use in the preventive or therapeutic treatment of vasculitis, or of a disease characterized by a M1/M2 macrophage ratio dysregulation selected from the group consisting of atherosclerosis, endometriosis, hypertension, osteonecrosis, Parkinson's disease, steatohepatitis, obesity-induced pathologies, lipodystrophy and myocardial infarction.

According to an embodiment, said composition is a pharmaceutical composition further comprising a pharmaceutically acceptable excipient.

According to an embodiment, said composition is a vaccine composition further comprising at least one adjuvant.

An object of the invention is a composition comprising a polypeptide as described herein, or a nucleic acid or a vector as described herein, for use in the preventive or therapeutic treatment of vasculitis.

According to an embodiment, said composition is a pharmaceutical composition further comprising a pharmaceutically acceptable excipient.

According to an embodiment, said composition is a vaccine composition further comprising at least one adjuvant.

According to another embodiment, the polypeptide is for use in simultaneous, separate or sequential combination with at least one adjuvant.

Another object of the invention is a kit-of-parts comprising a polypeptide as described herein and at least one adjuvant, for simultaneous, separate or sequential use in the preventive or therapeutic treatment of vasculitis, or of a disease characterized by a M1/M2 macrophage ratio dysregulation selected from the group consisting of atherosclerosis, endometriosis, hypertension, osteonecrosis, Parkinson's disease, steatohepatitis, obesity-induced pathologies, lipodystrophy and myocardial infarction.

An object of the invention is a kit-of-parts comprising a polypeptide as described herein and at least one adjuvant, for simultaneous, separate or sequential use in the preventive or therapeutic treatment of vasculitis.

According to an embodiment, said adjuvant is a natural or non-natural aluminum salt.

Another object of the invention is a method for decreasing the M1-type immune response and/or increasing the M2-type immune response, in a subject in need thereof, comprising administering to said subject a therapeutically effective amount of a polypeptide comprising, or consisting of, an amino acid sequence selected from the group consisting of:
a) the sequence of SEQ ID NO: 1, SEQ ID NO: 2, SEQ ID NO: 3, SEQ ID NO: 5 SEQ ID NO: 6, SEQ ID NO: 7 or SEQ ID NO: 8;
b) a fragment of a sequence defined in a), provided that said polypeptide decreases the M1-type immune response and/or increases the M2-type immune response; and
c) a sequence having at least 80% of identity with a sequence defined in a) or b), provided that said polypeptide decreases the M1-type immune response and/or increases the M2-type immune response.

According to an embodiment, the subject suffers from vasculitis, atherosclerosis, endometriosis, hypertension, osteonecrosis, Parkinson's disease, steatohepatitis, obesity-induced pathologies, lipodystrophy, or myocardial infarction.

According to an embodiment, said vasculitis is selected from the group consisting of Behçet's disease (BD), Cogan's syndrome (CS), Takayasu arteritis (TAK), Giant cell arteritis (GCA), Polyarteritis nodosa (PAN), Kawasaki disease (KD), Antineutrophil cytoplasmic antibody (ANCA)-associated vasculitis (AAV), Microscopic polyangiitis (MPA), Granulomatosis with polyangiitis (Wegener's) (GPA), Eosinophilic granulomatosis with polyangiitis (Churg-Strauss) (EGPA)), Immune complex small vessel vasculitis, Anti-glomerular basement membrane (anti-GBM) disease, Cryoglobulinemic vasculitis (CV), IgA vasculitis (Henoch-Schanlein) (IgAV), Hypocomplementemic urticarial vasculitis (HUV) (anti-C1q vasculitis), Cutaneous leukocytoclastic angiitis, Cutaneous arteritis, Primary central nervous system vasculitis, Isolated aortitis.

According to an embodiment, said vasculitis is Behçet's disease.

According to an embodiment, said vasculitis is associated to another disease selected from the group consisting of Lupus, Rheumatoid arthritis, Sarcoidosis, Hepatitis C, Hepatitis B, Syphilis and Cancer.

### Definitions

In the present invention, the following terms have the following meanings:

“Adjuvant”

As used herein, an “adjuvant” is a substance that enhances the immunogenicity of an immunogenic product of this invention. Adjuvants are often given to boost the immune response and are well known to the skilled artisan.

“Isolated”

As used herein, the term “isolated” or “non-naturally occurring” with reference to a biological component (such as a nucleic acid molecule, protein organelle or cells), refers to a biological component altered or removed from the natural state. For example, a nucleic acid or a peptide naturally present in a living animal is not “isolated” but the same nucleic acid or peptide partially or completely separated from the coexisting materials of its natural state is “isolated”. An isolated nucleic acid or peptide can exist in substantially purified form, or can exist in a non-native environment such as, for example, a host cell. Typically, a preparation of isolated nucleic acid or peptide contains the nucleic acid or peptide at least about 80% pure, at least about 85% pure, at least about 90% pure, at least about 95% pure, greater than 95% pure, greater than about 96% pure, greater than about 97% pure, greater than about 98% pure, or greater than about 99% pure. Nucleic acids and proteins that are “non-naturally occurring” or have been “isolated” include nucleic acids and proteins purified by standard purification methods. The term also embraces nucleic acids and proteins prepared by recombinant expression in a host cell as well as chemically synthesized nucleic acids. An “isolated polypeptide” is one that has been identified and separated and/or recovered from a component of its natural environment.

“Prevent” or “preventing” or “prevention”

As used herein, the terms “prevent”, “preventing” and “prevention” refer to prophylactic and preventive measures, wherein the object is to reduce the chances that a subject develop the pathologic condition or disorder over a given period of time. Such a reduction may be reflected, e.g., in a delayed onset of at least one symptom of the pathologic condition or disorder in the subject.

“Subject”

As used herein, the term “subject” refers to a warm-blooded animal, preferably a mammal. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human. Said human may be an adult or a child. A “child” is defined as an individual aged between 0 to 18 years. In one embodiment, a subject may be a “patient”, i.e., a subject who/which is awaiting the receipt of, or is receiving medical care or was/is/will be the object of a medical procedure, or is monitored for the development of a disease.

“Treating” or “treatment” or “alleviation”

As used herein, the terms “treating” or “treatment” or “alleviation” refer to therapeutic treatment, excluding prophylactic or preventive measures; wherein the object is to slow down (lessen) the targeted pathologic condition or disorder. Those in need of treatment include those already with the disorder as well those suspected to have the disorder. A subject is successfully “treated” for the targeted pathologic condition or disorder if, after receiving a therapeutic amount of the isolated polypeptide, nucleic acid, expression vector, composition, pharmaceutical composition or medicament according to the present invention, said subject shows observable and/or measurable reduction in or absence of one or more of the symptoms associated with the specific disease or condition, reduced morbidity and mortality, and/or improvement in quality of life issues. The above parameters for assessing successful treatment and improvement in the disease are readily measurable by routine procedures familiar to a physician.

### DETAILED DESCRIPTION

The 28 kDa and 26 kDa glutathione S-transferase native proteins are proteins expressed by the schistosome parasites, flatworms responsible for schistosomiasis. There are several species of schistosomes. Schistosoma mansoni is responsible for intestinal schistosomiasis in humans, in Africa and Brazil. Schistosoma haematobium is responsible for urinary schistosomiasis in humans, in Africa and the Arabian Peninsula. Each schistosome species expresses its own characteristic 28 kDa glutathione S-transferase. Thus, the species Schistosoma mansoni expresses Sm28GST and Sm26GST (two isoforms), the species Schistosoma haematobium expresses Sh28GST, Schistosoma bovis (a schistosome infecting livestock) expresses Sb28GST and the species Schistosoma japonicum (affecting South East Asia—the Philippines and South China) expresses Sj28GST and Sj26GST. The genes encoding these proteins are known and/or a person skilled in the art is capable of identifying them. A person skilled in the art is therefore able to produce the aforementioned proteins and polypeptides of the invention for instance by recombinant techniques.

The Sh28GST, Sm28GST, Sb28GST, Sj28GST, Sm26GST and Sj26GST proteins have sequences that are identified and listed in the databases. In particular, in the NCBI databases (https://www.ncbi.nlm.nih.gov), the polypeptide sequence of Sh28GST may be found under accession number XP_012797862, the polypeptide sequence of Sm28GST may be found under accession number XP_018646799, the polypeptide sequence of Sb28GST may be found under accession number AAA29893, the polypeptide sequence of Sj28GST may be found under accession number AAB03573, the polypeptide sequences of Sm26GST (isoforms 1 and 2) may be found under accession numbers AAA29888 and XP_018652834 respectively, and the polypeptide sequence of Sj26GST may be found under accession number AAB59203, as updated as of Apr. 9, 2020.

The Sh28GST and Sb28GST proteins are 97% identical, whereas the Sh28GST and Sm28GST proteins are 91% identical and the Sh28GST and Sj28GST proteins are 78% identical.

The sequences of the 28 kDa and 26 kDa glutathione S-transferase proteins from the various schistosomes are represented by the sequences of SEQ ID NO: 1, SEQ ID NO: 2, SEQ ID NO: 3, SEQ ID NO: 5, SEQ ID NO: 6, SEQ ID NO: 7 and SEQ ID NO: 8, which represent the sequences of the Sh28GST, Sm28GST, Sb28GST, Sj28GST, Sm26GST and Sj26GST proteins, respectively (see Table 1 below).

TABLE 1
Sequences of the Sh28GST, Sm28GST, Sb28GST, Sj28GST, Sm26GST and
Sj26GST proteins.

| | | |
|---|---|---|
| SEQ | Schistosoma | MTGDHIKVIYFNGRGRAESIRMTLVAAGVNYEDERISFQDWP |
| ID | haematobium | KIKPTIPGGRLPAVKITDNHGHVKWMVESLAIARYMAKKHH |
| NO: | Sh28GST | MMGGTEEEYYNVEKLIGQAEDLEHEYYKTLMKPEEEKQKIIK |
| 1 | | EILNGKVPVLLDIICESLKASTGKLAVGDKVTLADLVLIAVIDH |
| | | VTDLDKEFLTGKYPEIHKHRENLLASSPRLAKYLSDRAATPF |
| SEQ | Schistosoma | MAGEHIKVIYFDGRGRAESIRMTLVAAGVDYEDERISFQDWP |
| ID | mansoni | KIKPTIPGGRLPAVKVTDDHGHVKWMLESLAIARYMAKKHH |
| NO: | Sm28GST | MMGETDEEYYSVEKLIGQAEDVEHEYHKTLMKPQEEKEKIT |
| 2 | | KEILNGKVPVLLNMICESLKGSTGKLAVGDKVTLADLVLIAVI |
| | | DHVTDLDKGFLTGKYPEIHKHRENLLASSPRLAKYLSNRPATP |
| | | F |
| SEQ | Schistosoma | MTGDHIKVIYFNGRGRAESIRMTLVAAGVNYEDERISFQDWP |
| ID | bovis | KIKPTIPGGRLPAVKITDNHGHVKWMLESLAIARYMAKKHH |
| NO: | Sb28GST | MMGETDEEYYNVEKLIGQVEDLEHEYHKTLMKPEEEKQKIT |
| 3 | | KEILNGKVPVLLDIICESLKASTGKLAVGDKVTLADLVLIAVID |
| | | HVTDLDKEFLTGKYPEIHKHRENLLASSPRLAKYLSDRAATPF |
| SEQ | Schistosoma | MACGHVKLIYFNGRGRAEPIRMILVAAGVEFEDERIEFQDWP |
| ID | japonicum | KIKPTIPGGRLPIVKITDKRGDVKTMSESLAIARFIARKHNMM |
| NO: | Sj28GST | GDTDDEYYIIEKMIGQVEDVESEYHKTLMKPPEEKEKISKEIL |
| 5 | | NGKVPILLQAICETLKESTGNLTVGDKVTLADVVLIASIDHITD |
| | | LDKEFLTGKYPEIHKHRKHLLATSPKLAKYLSERHATAF |
| SEQ | Schistosoma | MAPKFGYWKVKGLVQPTRLLLEHLEETYEERAYDRNEIDAW |
| ID | mansoni | SNDKFKLGLEFPNLPYYIDGDFKLTQSMAIIRYIADKHNMLGA |
| NO: | Sm26GST | CPKERAEISMLEGAVLDIRMGVLRIAYNKEYETLKVDFLNKL |
| 6 | isoform 1 | PGRLKMFEDRLSNKTYLNGNCVTHPDFMLYDALDVVLYMDS |
| | | QCLNEFPKLVSFKKCIEDLPQIKNYLNSSRYIKWPLQGWDATF |
| | | GGGDTPPK |
| SEQ | Schistosoma | MAPKLGYWKIKGLVQPTRLLLEYLGEAYEERLYDRNDGDV |
| ID | mansoni | WRNEKFKLGLDFPNLPYYIDGDVKLTQSMAILRYIADKHNML |
| NO: | Sm26GST | GGCPKERAEISMLEGAILDIRYGVSRIAYNKEFETLKVDFLNQ |
| 7 | isoform 2 | LPGMLKMFEDRLSHNTYLNGDKVTHPDFMLYDALDVNLPPI |
| | | KNYLNSNRYIKWPLQGWSATFGGGDAPPK |
| SEQ | Schistosoma | MSPILGYWKIKGLVQPTRLLLEYLEEKYEEHLYERDEGDKWR |
| ID | japonicum | NKKFELGLEFPNLPYYIDGDVKLTQSMAIIRYIADKHNMLGG |
| NO: | Sj26GST | CPKERAEISMLEGAVLDIRYGVSRIAYSKDFETLKVDFLSKLP |
| 8 | | EMLKMFEDRLCHKTYLNGDHVTHPDFMLYDALDVVLYMDP |
| | | MCLDAFPKLVCFKKRIEAIPQIDKYLKSSKYIAWPLQGWQATF |
| | | GGGDHPPK |

In some embodiments, the polypeptide of the invention comprises, or consists of, an amino acid sequence selected from the group consisting of SEQ ID NO: 1, SEQ ID NO: 2, SEQ ID NO: 3, SEQ ID NO: 5, SEQ ID NO: 6, SEQ ID NO: 7 and SEQ ID NO: 8.

In some embodiments, the polypeptide of the invention is an isolated polypeptide.

The polypeptides of the invention are capable of reducing or suppressing the inflammatory reaction (in particular, that mediated by M1 macrophages) and/or of inducing an anti-inflammatory immune response (for example mediated by M2 macrophages).

Indeed, the inventors have surprisingly shown that P28GST proteins from Schistosoma are capable of reducing the M1-type macrophage immune response. This decrease in the M1-type immune response allows reducing the symptoms associated with inflammation observed in vasculitis.

A particular type of immune response is characterized by various aspects such as e.g. the type of immune cells, of cytokines, of immune mediators, etc., that are involved in said immune response.

As used herein, the “M1-type immune response” or “M1-type macrophage immune response” denotes an immune response at least partly mediated by “M1-type macrophages” and/or molecules produced by “M1-type macrophages”.

Similarly, the “M2-type immune response” or “M2-type macrophage immune response” denotes an immune response at least partly mediated by “M2-type macrophages” and/or molecules produced by “M2-type macrophages”.

The “M1-type immune response” and “M2-type immune response” are well known in the art and are reviewed for instance in Ansari (2015) Journal of the Neurological Sciences 357:41-49 and in Xin et al. (2016) Biochemical and Biophysical Research Communications 477:589-594.

As shown in the Example section, the polypeptides of the invention decrease the secretion of pro-inflammatory cytokines and/or mediators by M1 macrophages and/or increase the secretion of anti-inflammatory cytokines and/or mediators by M2 macrophages.

The polypeptides of the invention thus have a biological activity.

In some embodiment, the “biological activity” of the polypeptides of the invention denotes an activity that decreases the M1-type immune response and/or increases the M2-type immune response.

In particular, “decreasing the M1-type immune response and/or increasing the M2-type immune response” may for instance mean decreasing the number of M1 macrophages, increasing the number of M2 macrophages, decreasing the M1/M2 macrophage ratio, increasing the M2/M1 macrophage ratio, decreasing the secretion of pro-inflammatory cytokines or mediators (e.g. by M1 macrophages), decreasing the level of pro-inflammatory cytokines or mediators, increasing the secretion of anti-inflammatory cytokines or mediators (e.g. by M2 macrophages), and/or increasing the level of pro-inflammatory cytokines or mediators.

A polypeptide of the invention has a biological activity within the meaning of the present invention as soon as it has at least one of the above-mentioned activities.

As used herein, the term “M1 macrophage” or “classical macrophage” refers to a subset of pro-inflammatory macrophages, i.e. macrophages causing inflammation. M1 macrophage subset is typically activated by microbial products such as lipopolysaccharide (LPS), or pro-inflammatory cytokines such as interferon-γ. They usually kill pathogens by releasing reactive oxygen and nitrogen species as well as pro-inflammatory cytokines or mediators.

As used herein, the term “M2 macrophage” or “alternatively activated macrophage” refers to a subset of anti-inflammatory macrophages or regulatory macrophages, which are involved in the resolution of inflammation, angiogenesis, tissue remodeling, and repair. These macrophages are stimulated by cytokines like IL-4, IL-10, or IL-13 and, in turn, produce anti-inflammatory cytokines or mediators.

As used herein, the term “pro-inflammatory cytokines or mediators” includes, without being limited to, cytokines or interleukins such as e.g. IL-1, IL-1β, IL-6, IL-12, IL-15, IL-17, IL-23, TNF-α, IFN-γ, S100 proteins, serum amyloid A (SAA) or oncostatin M.

As used herein, the term “anti-inflammatory cytokines or mediators” includes, without being limited to, cytokines or interleukins such as e.g. IL-10, IL-37, macrophage colony stimulating factor (M-CSF), transforming growth factor (TGF)-β or mouse Chitinase-3-like 3.

The biological activity of a polypeptide can easily be assessed in vitro, ex vivo or in vivo, by persons skilled in the art, in particular by means of the following assays.

For instance, the number of M1 macrophages (respectively M2 macrophages) may be measured by flow cytometry or by quantitative PCR (qPCR), preferably real time quantitative PCR (RT-qPCR).

For instance, these assays may be used to detect and quantify biomarkers which are M1-specific biomarkers which may be surface biomarkers such as e.g. CD80, CD86, or intracellular biomarkers such as e.g. iNOS, Cox2, STAT-1, IRF5.

Also, as an example, these assays may be used to detect and quantify biomarkers which are M2-specific biomarkers which may be surface biomarkers such as e.g. mouse CD200R, CD206, CD163, or intracellular biomarkers such as e.g. mouse Arg-1, PPARγ, STAT-6, IRF4.

As a non-limiting example, the flow cytometry assay done to determine the number of M1 macrophages (respectively M2 macrophages) may be performed as follows. Typically, cells may be blocked and viability assessed using fixable viability dye. Then, cell surface immunostaining may typically be performed using antibodies such as e.g. antibodies directed against CD80, CD86, CD206, CD200R or CD163 . . . . Typically, cells may then be analyzed on a flow cytometer and data analysis may for instance be performed using appropriate Software. As a non-limiting example of flow cytometry protocol is given in the Materials and Methods of Example 1.

As a non-limiting example, the qPCR assay done to determine the number of M1 macrophages (respectively M2 macrophages) may be performed as follows. Typically, total RNA may be extracted from cells, for instance cells of the skin. The RNA concentration and purity may typically be determined by the absorbance at 260 nm and 280 nm. Retro-transcription may typically be performed using an appropriate commercial kit known by the person skilled in the art. Gene expression may then be typically evaluated by RT-qPCR, for instance by using Fast SYBR Green Master Mix reagent on an appropriate instrument. Normalization may for instance be done with β-actin or any other housekeeping gene. Suitable primers may be used such as e.g. forward and reverse primers allowing detection of β-actin, iNOS, Arg1. Results may typically be expressed as relative expression compared to control, for instance using the 2-ΔΔCt method. As a non-limiting example of flow cytometry protocol is given in the Materials and Methods of Example 1.

The number of M1 macrophages may be measured at several timepoints, such as e.g. before and after administration of a polypeptide of the invention, and the measured numbers may then be compared in order to determine the evolution (decrease or increase) of the number of M1 macrophages following polypeptide administration.

Similarly, the number of M2 macrophages may be measured at several timepoints, such as e.g. before and after administration of a polypeptide of the invention, and the measured numbers may then be compared in order to determine the evolution (decrease or increase) of the number of M2 macrophages following polypeptide administration.

Alternatively, the number of M1 macrophages measured in the presence of, or after administration of, a polypeptide of the invention may be compared to the number of M1 macrophages measured in a negative control (e.g. in the absence of the polypeptide) in order to determine the effect (decrease or increase) of the polypeptide on the number of M1 macrophages.

Similarly, the number of M2 macrophages measured in the presence of, or after administration of, a polypeptide of the invention may be compared to the number of M2 macrophages measured in a negative control (e.g. in the absence of the polypeptide) in order to determine the effect (decrease or increase) of the polypeptide on the number of M2 macrophages.

To assess the biological activity of a polypeptide, the level of pro-inflammatory cytokines or mediators (respectively anti-inflammatory cytokines or mediators) may also be measured, for instance by ELISA, preferably by quantitative ELISA, Multiplex analysis, or by quantitative PCR, preferably real time quantitative PCR (RT-qPCR).

For instance, these assays may be used to detect and quantify pro-inflammatory cytokines or mediators such as e.g. IL-1, IL-1β, IL-6, IL-12, IL-15, IL-17, IL-23, TNF-α IFN-γ, S100 proteins, serum amyloid A (SAA) or oncostatin M, or to detect and quantify anti-inflammatory cytokines or mediators such as e.g. IL-10, IL-37, macrophage colony stimulating factor (M-CSF), transforming growth factor (TGF)-β or Chitinase-3-like 3.

As a non-limiting example, the qPCR assay done to quantify the level of a cytokine or mediator may be performed as follows. Typically, total RNA may be extracted from cells, for instance cells of the skin. The RNA concentration and purity may typically be determined by the absorbance at 260 nm and 280 nm. Retro-transcription may typically be performed using an appropriate commercial kit known by the person skilled in the art. Gene expression may then be typically evaluated by RT-qPCR, for instance by using Fast SYBR Green Master Mix reagent on an appropriate instrument. Normalization may for instance be done with β-actin or any other housekeeping gene. Suitable primers may be used such as e.g. forward and reverse primers allowing detection of IL-1β, TNFα, β-actin. Results may typically be expressed as relative expression compared to control, for instance using the 2-ΔΔCt method. A non-limiting example of flow cytometry protocol is given in the Materials and Methods of Example 1.

As a non-limiting example, an ELISA assay done to quantify the level of cytokines or mediator such as C-reactive protein, S100A8 or S100A9 protein may be performed as follows. Typically, the test kit may for instance be a solid phase enzyme immunometric assay (ELISA) in the microplate format, designed for the quantitative measurement of target molecule. The microplate may typically be coated with a capture antibody. Then, calibrators and samples may typically be added e.g. for 2 hours of incubation. During this incubation, endogenous target in the sample bound to the antibodies may fix onto the inner surface of the wells. Non-reactive sample components may typically be removed by a washing step. Afterwards, a biotinylated detection antibody may for instance be added. During e.g. a 2-hour incubation, a sandwich complex consisting of the two antibodies and the target may form. Excess of detection antibody may typically be washed out. Then, streptavidin conjugated to horseradish-peroxidase may typically be added to complete the sandwich for e.g. 20 minutes of incubation. Excess of enzyme conjugate may typically be washed out. Finally, a chromogenic substrate, such as e.g. TMB (3,3′,5,5′-Tetra-Methyl-Benzidine) may typically be added to all wells. During e.g. 20 minutes of incubation, the substrate may typically be converted to a colored end product by the fixed enzyme. Enzyme reaction may typically be stopped by dispensing of, for instance, hydrochloric acid as stop solution. In these conditions, the color intensity would be directly proportional to the concentration of the target present in the sample. The optical density of the color solution may typically be measured for instance with a microplate reader at 450 nm. A non-limiting example of multiplex analysis protocol is given in the Materials and Methods of Example 2.

The level of pro-inflammatory cytokines or mediators (respectively anti-inflammatory cytokines or mediators) may be measured at several timepoints, such as e.g. before and after administration of a polypeptide of the invention, and the measured levels may then be compared in order to determine the evolution (decrease or increase) of the level of pro-inflammatory cytokines or mediators (respectively anti-inflammatory cytokines or mediators) following polypeptide administration.

Alternatively, the level of pro-inflammatory cytokines or mediators (respectively anti-inflammatory cytokines or mediators) measured in the presence of, or after administration of, a polypeptide of the invention may be compared to the level of pro-inflammatory cytokines or mediators (respectively anti-inflammatory cytokines or mediators) measured in a negative control (e.g. in the absence of the polypeptide) in order to determine the effect (decrease or increase) of the polypeptide on the level of pro-inflammatory cytokines or mediators (respectively anti-inflammatory cytokines or mediators).

A first measured value, number or level is herein considered to be decreased compared to a second measured value, number or level, if the first measured value, number or level 5 is at least 1%, 5%, 10%, 15%, 20%, 25%, 30%, 40%, 50%, 60%, 70%, 80%, 90%, 100%, 150%, 200% or 300% lower than second measured value, number or level.

A first measured value, number or level is herein considered to be increased compared to a second measured value, number or level, if the first measured value, number or level is at least 1%, 5%, 10%, 15%, 20%, 25%, 30%, 40%, 50%, 60%, 70%, 80%, 90%, 100%, 150%, 200% or 300% higher than the second measured value, number or level.

Preferably, a first measured value, number or level is herein considered to be decreased compared to a second measured value, number or level, if the first measured value, number or level is statistically lower than the second measured value, number or level, i.e. if the p-value is less than 0.05 in the appropriate statistical test.

Preferably, a first measured value, number or level is herein considered to be increased compared to a second measured value, number or level, if the first measured value, number or level is statistically higher than the second measured value, number or level, i.e. if the p-value is less than 0.05 in the appropriate statistical test

In some embodiments, the “first measured value, number or level” is a value, number or level measured in the presence of, or after administration of, a polypeptide of the invention, whereas the “second measured value, number or level” is a value, number or level measured in the absence of, or before administration of, the polypeptide.

In some embodiments, the polypeptide of the invention comprises, or consists of, a fragment of an amino acid sequence selected from the group consisting of SEQ ID NO: 1, SEQ ID NO: 2, SEQ ID NO: 3, SEQ ID NO: 5, SEQ ID NO: 6, SEQ ID NO: 7 and SEQ ID NO: 8.

In one embodiment, the polypeptide of the invention comprises, or consists of, a fragment of the amino acid sequence of SEQ ID NO: 1.

In one embodiment, the polypeptide of the invention comprises, or consists of, a fragment of the amino acid sequence of SEQ ID NO: 2.

In one embodiment, the polypeptide of the invention comprises, or consists of, a fragment of the amino acid sequence of SEQ ID NO: 3.

In one embodiment, the polypeptide of the invention comprises, or consists of, a fragment of the amino acid sequence of SEQ ID NO: 5.

In one embodiment, the polypeptide of the invention comprises, or consists of, a fragment of the amino acid sequence of SEQ ID NO: 6.

In one embodiment, the polypeptide of the invention comprises, or consists of, a fragment of the amino acid sequence of SEQ ID NO: 7.

In one embodiment, the polypeptide of the invention comprises, or consists of, a fragment of the amino acid sequence of SEQ ID NO: 8.

By “fragment” of a reference sequence is meant herein a sequence constituted by a chain of consecutive amino acids of a reference sequence and whose size is smaller than the size of the reference sequence. In the context of the invention, the fragments may for example have a size of between 6 and 210, 6 and 200, 6 and 175, 6 and 150, 6 and 125, 6 and 100, 6 and 75, 6 and 50, 6 and 25, 6 and 15, 6 and 10 amino acids, or a size of between 6 and 210, 10 and 210, 25 and 210, 50 and 210, 75 and 210, 100 and 210, 125 and 210, 150 and 210, 175 and 210, 200 and 210, 205 and 210 amino acids.

According to one embodiment, the fragment according to the invention comprises at least one fragment having an amino acid sequence selected from the group consisting of the sequence ranging from the amino acid at position 15 to the amino acid at position 60, the sequence ranging from the amino acid at position 20 to the amino acid at position 50, the sequence ranging from the amino acid at position 24 to the amino acid at position 43 of SEQ ID NO: 1, SEQ ID NO: 2, SEQ ID NO: 3 or SEQ ID NO: 5.

According to one embodiment, the fragment according to the invention comprises at least one fragment having an amino acid sequence selected from the group consisting of the sequence ranging from the amino acid at position 100 to the amino acid at position 150, the sequence ranging from the amino acid at position 110 to the amino acid at position 140, the sequence ranging from the amino acid at position 115 to the amino acid at position 131 of SEQ ID NO: 1, SEQ ID NO: 2, SEQ ID NO: 3 or SEQ ID NO: 5.

According to another embodiment, the fragment according to the invention comprises at least one fragment having an amino acid sequence selected from the group consisting of the sequence ranging from the amino acid at position 170 to the amino acid at position 211, the sequence ranging from the amino acid at position 180 to the amino acid at position 211, or the sequence ranging from the amino acid at position 190 to the amino acid at position 211 of SEQ ID NO: 1, SEQ ID NO: 2, SEQ ID NO: 3 or SEQ ID NO: 5.

According to one embodiment, the fragment according to the invention comprises at least one fragment having an amino acid sequence selected from the group consisting of the sequence ranging from the amino acid at position 10 to the amino acid at position 60, the sequence ranging from the amino acid at position 15 to the amino acid at position 50, the sequence ranging from the amino acid at position 21 to the amino acid at position 43 of SEQ ID NO: 6, SEQ ID NO: 7 or SEQ ID NO: 8.

According to one embodiment, the fragment according to the invention comprises at least one fragment having an amino acid sequence selected from the group consisting of the sequence ranging from the amino acid at position 100 to the amino acid at position 140, the sequence ranging from the amino acid at position 105 to the amino acid at position 130, the sequence ranging from the amino acid at position 112 to the amino acid at position 125 of SEQ ID NO: 6, SEQ ID NO: 7 or SEQ ID NO: 8.

According to one embodiment, the fragment according to the invention comprises at least one fragment having an amino acid sequence selected from the group consisting of the sequence ranging from the amino acid at position 160 to the amino acid at position 218, the sequence ranging from the amino acid at position 170 to the amino acid at position 217, the sequence ranging from the amino acid at position 181 to the amino acid at position 217 of SEQ ID NO: 6.

According to one embodiment, the fragment according to the invention comprises at least one fragment having an amino acid sequence selected from the group consisting of the sequence ranging from the amino acid at position 150 to the amino acid at position 195, the sequence ranging from the amino acid at position 160 to the amino acid at position 190, the sequence ranging from the amino acid at position 172 to the amino acid at position 187 of SEQ ID NO: 7.

According to one embodiment, the fragment according to the invention comprises at least one fragment having an amino acid sequence selected from the group consisting of the sequence ranging from the amino acid at position 160 to the amino acid at position 218, the sequence ranging from the amino acid at position 170 to the amino acid at position 210, the sequence ranging from the amino acid at position 181 to the amino acid at position 203 of SEQ ID NO: 8.

According to some embodiments, the fragment according to the invention comprises:
at least one fragment having an amino acid sequence selected from the group consisting of the sequence ranging from the amino acid at position 15 to the amino acid at position 60, the sequence ranging from the amino acid at position 20 to the amino acid at position 50, the sequence ranging from the amino acid at position 24 to the amino acid at position 43 of SEQ ID NO: 1, SEQ ID NO: 2, SEQ ID NO: 3 or SEQ ID NO: 5,
at least one fragment having an amino acid sequence selected from the group consisting of the sequence ranging from the amino acid at position 100 to the amino acid at position 150, the sequence ranging from the amino acid at position 110 to the amino acid at position 140, the sequence ranging from the amino acid at position 115 to the amino acid at position 131 of SEQ ID NO: 1, SEQ ID NO: 2, SEQ ID NO: 3 or SEQ ID NO: 5, and
at least one fragment having an amino acid sequence selected from the group consisting of the sequence ranging from the amino acid at position 170 to the amino acid at position 211, the sequence ranging from the amino acid at position 180 to the amino acid at position 211, or the sequence ranging from the amino acid at position 190 to the amino acid at position 211 of SEQ ID NO: 1, SEQ ID NO: 2, SEQ ID NO: 3 or SEQ ID NO: 5.

According to some embodiments, the fragment according to the invention comprises:
at least one fragment having an amino acid sequence selected from the group consisting of the sequence ranging from the amino acid at position 10 to the amino acid at position 60, the sequence ranging from the amino acid at position 15 to the amino acid at position 50, the sequence ranging from the amino acid at position 21 to the amino acid at position 43 of SEQ ID NO: 6, SEQ ID NO: 7 or SEQ ID NO: 8,
at least one fragment having an amino acid sequence selected from the group consisting of the sequence ranging from the amino acid at position 100 to the amino acid at position 140, the sequence ranging from the amino acid at position 105 to the amino acid at position 130, the sequence ranging from the amino acid at position 112 to the amino acid at position 125 of SEQ ID NO: 6, SEQ ID NO: 7 or SEQ ID NO: 8, and
at least one fragment having an amino acid sequence selected from the group consisting of the sequence ranging from the amino acid at position 181 to the amino acid at position 217 of SEQ ID NO: 6, or the sequence ranging from the amino acid at position 172 to the amino acid at position 187 of SEQ ID NO: 7, or the sequence ranging from the amino acid at position 181 to the amino acid at position 203 of SEQ ID NO: 8.

The polypeptides of the invention also include any polypeptide which is a “variant”, “homologue” or “derivative” of the hereabove polypeptides and which exhibits the same biological activity.

Preferably, the polypeptides of the invention are variants of the native 28 kDa glutathione S-transferase proteins from Schistosoma haematobium, Schistosoma mansoni, Schistosoma bovis or Schistosoma japonicum, or variants of the native 26 kDa glutathione S-transferase proteins from Schistosoma mansoni or Schistosoma japonicum.

The polypeptides of the invention thus include polypeptides having sequences derived from the amino acid sequence of SEQ ID NO: 1, or derived from fragments of the amino acid sequence of SEQ ID NO: 1, defined by a percentage of sequence identity with the sequence of SEQ ID NO: 1.

The polypeptides of the invention also include polypeptides having sequences derived from the amino acid sequence of SEQ ID NO: 2, or derived from fragments of the amino acid sequence of SEQ ID NO: 2, defined by a percentage of sequence identity with the sequence of SEQ ID NO: 2.

The polypeptides of the invention further include polypeptides having sequences derived from the amino acid sequence of SEQ ID NO: 3, or derived from fragments of the amino acid sequence of SEQ ID NO: 3, defined by a percentage of sequence identity with the sequence of SEQ ID NO: 3.

The polypeptides of the invention also include polypeptides having sequences derived from the amino acid sequence of SEQ ID NO: 5, or derived from fragments of the amino acid sequence of SEQ ID NO: 5, defined by a percentage of sequence identity with the sequence of SEQ ID NO: 5.

The polypeptides of the invention further include polypeptides having sequences derived from the amino acid sequence of SEQ ID NO: 6, or derived from fragments of the amino acid sequence of SEQ ID NO: 6, defined by a percentage of sequence identity with the sequence of SEQ ID NO: 6.

The polypeptides of the invention further include polypeptides having sequences derived from the amino acid sequence of SEQ ID NO: 7, or derived from fragments of the amino acid sequence of SEQ ID NO: 7, defined by a percentage of sequence identity with the sequence of SEQ ID NO: 7.

The polypeptides of the invention further include polypeptides having sequences derived from the amino acid sequence of SEQ ID NO: 8, or derived from fragments of the amino acid sequence of SEQ ID NO: 8, defined by a percentage of sequence identity with the sequence of SEQ ID NO: 8.

The “variant”, “homologue” or “derivative” polypeptides are defined as comprising a sequence identical to at least 80%, preferably at least 85%, more preferably at least 90%, even at least 95%, 96%, 97%, 98% or 99% of the reference sequence.

These derived sequences may differ from the reference sequence by substitution, deletion and/or insertion of one or more amino acids, at positions such that these modifications do not have any significant impact on the biological activity of the polypeptides.

The substitutions may in particular correspond to conservative substitutions or to substitutions of natural amino acids by non-natural amino acids or pseudo amino acids.

By “amino acid sequence having (for instance) at least 80% of identity with a reference sequence” is meant herein a sequence identical to the reference sequence but this sequence may comprise up to twenty mutations (substitutions, deletions and/or insertions) per each part of one hundred amino acids of the reference sequence. Therefore, for a reference sequence of 100 amino acids, a fragment of 80 amino acids and a sequence of 100 amino acids comprising 20 substitutions compared with the reference sequence are two examples of sequences having 80% sequence identity with the reference sequence.

Percentage of identity is generally determined using sequence analysis software (for example the Sequence Analysis Software Package of the Genetics Computer Group, University of Wisconsin Biotechnology Center, 1710 University Avenue, Madison, Wis. 53705). The amino acid sequences to be compared are aligned to obtain maximum percentage identity. For this purpose, it may be necessary to artificially add gaps in the sequence. The alignment can be performed manually or automatically. Automated alignment algorithms of nucleotide sequences are well known to persons skilled in the art and described for example in Altschul et al. (1997) Nucleic Acids Res. 25:3389 and implemented by softwares such as the BLAST software. One algorithm which can be isolated is the Needleman-Wunsch algorithm for example (Needleman and Wunsch (1970) J Mol Biol. 48:443-53). Once optimal alignment has been achieved, the percentage identity is established by recording all the positions at which the amino acids of the two compared sequences are identical, compared with the total number of positions.

In one embodiment, the polypeptide of the invention comprises or consists of:

a) a sequence having at least 80%, 85%, 90%, 95% or 100% of identity with the amino acid sequence SEQ ID NO: 1, or

b) a sequence having at least 80%, 85%, 90%, 95% or 100% of identity with a fragment of the amino acid sequence SEQ ID NO: 1.

In one embodiment, the polypeptide of the invention comprises or consists of:

a) a sequence having at least 80%, 85%, 90%, 95% or 100% of identity with the amino acid sequence SEQ ID NO: 2, or

b) a sequence having at least 80%, 85%, 90%, 95% or 100% of identity with a fragment of the amino acid sequence SEQ ID NO: 2.

In one embodiment, the polypeptide of the invention comprises or consists of:

a) a sequence having at least 80%, 85%, 90%, 95% or 100% of identity with the amino acid sequence SEQ ID NO: 3, or

b) a sequence having at least 80%, 85%, 90%, 95% or 100% of identity with a fragment of the amino acid sequence SEQ ID NO: 3.

In one embodiment, the polypeptide of the invention comprises or consists of:

a) a sequence having at least 80%, 85%, 90%, 95% or 100% of identity with the amino acid sequence SEQ ID NO: 5, or

b) a sequence having at least 80%, 85%, 90%, 95% or 100% of identity with a fragment of the amino acid sequence SEQ ID NO: 5.

In one embodiment, the polypeptide of the invention comprises or consists of:

a) a sequence having at least 80%, 85%, 90%, 95% or 100% of identity with the amino acid sequence SEQ ID NO: 6, or

b) a sequence having at least 80%, 85%, 90%, 95% or 100% of identity with a fragment of the amino acid sequence SEQ ID NO: 6.

In one embodiment, the polypeptide of the invention comprises or consists of:

a) a sequence having at least 80%, 85%, 90%, 95% or 100% of identity with the amino acid sequence SEQ ID NO: 7, or

b) a sequence having at least 80%, 85%, 90%, 95% or 100% of identity with a fragment of the amino acid sequence SEQ ID NO: 7.

In one embodiment, the polypeptide of the invention comprises or consists of:

a) a sequence having at least 80%, 85%, 90%, 95% or 100% of identity with the amino acid sequence SEQ ID NO: 8, or

b) a sequence having at least 80%, 85%, 90%, 95% or 100% of identity with a fragment of the amino acid sequence SEQ ID NO: 8.

In one embodiment, the sequence of the polypeptides differs from the reference sequence solely through the presence of conservative substitutions. Conservative substitutions are substitutions of amino acids of the same class, such as substitutions of amino acids with non-charged side chains (such as asparagine, glutamine, serine, cysteine, and tyrosine), of amino acids with basic side chains (such as lysine, arginine and histidine), of amino acids with acid side chains (such as aspartic acid and glutamic acid), of amino acids with non-polar side chains (such as alanine, valine, leucine, isoleucine, proline, phenylalanine, methionine and tryptophan).

According to the invention, the polypeptides may be modified chemically or enzymatically to improve their stability or bioavailability. Such chemical or enzymatic modifications are well known to those skilled in the art. Mention may be made of the following modifications but they are not limited thereto:
modifications of the C-terminal or N-terminal end of the polypeptides such as N-terminal deamination or acylation (preferably acetylation) or such as C-terminal amidation or esterification;
modifications of the amide bond between two amino acids, such as acylation (preferably acetylation) or alkylation at the nitrogen or alpha carbon;
changes in chirality, such as the substitution of a natural amino acid (L-enantiomer) by the corresponding D-enantiomer; this modification may optionally be accompanied by inversion of the side chain (from the C-terminal end to the N-terminal end);
changes to azapeptides, in which one or more alpha carbons are replaced by nitrogen atoms; and/or
changes to betapeptides, in which one or more carbons are added on the N-alpha side or on the C-alpha side of the main chain.

In this respect, it is possible to modify one or more of the lysine amino acids (K) of the polypeptides, notably by:
amidation: this modification is simple to achieve, the positive charge of the lysine being substituted by hydrophobic groups (for example acetyl or phenylacetyl);
amination: by formation of secondary amide from the primary amine R═(CH₂)₄—NH₃⁺, for example by forming N-methyl, N-allyl or N-benzyl groups; and
by formation of N-oxide, N-nitroso, N-dialkyl phosphoryl, N-sulfenyl, or N-glycoside groups.

It is also or alternatively possible to modify one or more threonine (T) and/or serine (S) amino acids of the polypeptides, notably by adding an ester or ether group at the OH group of the side chain of threonine and/or serine. Esterification, a simple operation, can be performed using a carboxylic acid, an anhydride, by bridging, etc, to form acetates or benzoates. Etherification, which gives more stable compounds, can be performed using an alcohol, a halide, etc. to form a methyl ether for example or an O-glycoside.

It is also or alternatively possible to modify one or more glutamine (Q) amino acids for example by amidation, by forming secondary or tertiary amines, in particular with groups of methyl, ethyl type, whether or not functionalized.

It is also or alternatively possible to modify one or more glutamate (E) and/or aspartate (D) amino acids, for example:
by esterification, to form methyl esters, whether or not substituted, ethyl esters, benzyl esters, thiols (activated esters); and
by amidation, notably to form N,N dimethyl groups, nitroanilides, pyrrolidinyls.
On the other hand, it is preferable not to modify the proline amino acids, which take part in the secondary structure of the polypeptides, bearing also in mind that the amino acids G, A and M in general do not offer modification possibilities of clear interest.

According to an embodiment, the polypeptide of the invention has a sequence derived from or homologous to one of the sequences SEQ ID NO: 1, SEQ ID NO: 2, SEQ ID NO: 3 and SEQ ID NO: 5, which advantageously comprises at least one fragment having an amino acid sequence selected from the group consisting of the sequence ranging from the amino acid at position 15 to the amino acid at position 60, the sequence ranging from the amino acid at position 20 to the amino acid at position 50, the sequence ranging from the amino acid at position 24 to the amino acid at position 43 of SEQ ID NO: 1, SEQ ID NO: 2, SEQ ID NO: 3 or SEQ ID NO: 5.

According to an embodiment, the polypeptide of the invention has a sequence derived from or homologous to one of the sequences SEQ ID NO: 1, SEQ ID NO: 2, SEQ ID NO: 3 and SEQ ID NO: 5, which advantageously comprises at least one fragment having an amino acid sequence selected from the group consisting of the sequence ranging from the amino acid at position 100 to the amino acid at position 150, the sequence ranging from the amino acid at position 110 to the amino acid at position 140, the sequence ranging from the amino acid at position 115 to the amino acid at position 131 of SEQ ID NO: 1, SEQ ID NO: 2, SEQ ID NO: 3 or SEQ ID NO: 5.

According to an embodiment, the polypeptide of the invention has a sequence derived from or homologous to one of the sequences SEQ ID NO: 1, SEQ ID NO: 2, SEQ ID NO: 3 and SEQ ID NO: 5, which advantageously comprises at least one fragment having an amino acid sequence selected from the group consisting of the sequence ranging from the amino acid at position 170 to the amino acid at position 211, the sequence ranging from the amino acid at position 180 to the amino acid at position 211, or the sequence ranging from the amino acid at position 190 to the amino acid at position 211 of SEQ ID NO: 1, SEQ ID NO: 2, SEQ ID NO: 3 or SEQ ID NO: 5.

According to another embodiment, the polypeptide of the invention has a sequence derived from or homologous to one of the sequences SEQ ID NO: 6, SEQ ID NO: 7 and SEQ ID NO: 8, which advantageously comprises at least one fragment having an amino acid sequence selected from the group consisting of the sequence ranging from the amino acid at position 10 to the amino acid at position 60, the sequence ranging from the amino acid at position 15 to the amino acid at position 50, the sequence ranging from the amino acid at position 21 to the amino acid at position 43 of SEQ ID NO: 6, SEQ ID NO: 7 or SEQ ID NO: 8.

According to another embodiment, the polypeptide of the invention has a sequence derived from or homologous to one of the sequences SEQ ID NO: 6, SEQ ID NO: 7 and SEQ ID NO: 8, which advantageously comprises at least one fragment having an amino acid sequence selected from the group consisting of the sequence ranging from the amino acid at position 100 to the amino acid at position 140, the sequence ranging from the amino acid at position 105 to the amino acid at position 130, the sequence ranging from the amino acid at position 112 to the amino acid at position 125 of SEQ ID NO: 6, SEQ ID NO: 7 or SEQ ID NO: 8.

According to another embodiment, the polypeptide of the invention has a sequence derived from or homologous to the sequence SEQ ID NO: 6, which advantageously comprises at least one fragment having an amino acid sequence selected from the group consisting of the sequence ranging from the amino acid at position 160 to the amino acid at position 218, the sequence ranging from the amino acid at position 170 to the amino acid at position 217, the sequence ranging from the amino acid at position 181 to the amino acid at position 217 of SEQ ID NO: 6.

According to another embodiment, the polypeptide of the invention has a sequence derived from or homologous to the sequence SEQ ID NO: 7, which advantageously comprises at least one fragment having an amino acid sequence selected from the group consisting of the sequence ranging from the amino acid at position 150 to the amino acid at position 195, the sequence ranging from the amino acid at position 160 to the amino acid at position 190, the sequence ranging from the amino acid at position 172 to the amino acid at position 187 of SEQ ID NO: 7.

According to another embodiment, the polypeptide of the invention has a sequence derived from or homologous to the sequence SEQ ID NO: 8, which advantageously comprises at least one fragment having an amino acid sequence selected from the group consisting of the sequence ranging from the amino acid at position 160 to the amino acid at position 218, the sequence ranging from the amino acid at position 170 to the amino acid at position 210, the sequence ranging from the amino acid at position 181 to the amino acid at position 203 of SEQ ID NO: 8.

According to some embodiments, the polypeptide of the invention has a sequence derived from or homologous to one of the sequences SEQ ID NO: 1, SEQ ID NO: 2, SEQ ID NO: 3 and SEQ ID NO: 5, which advantageously comprises:
at least one fragment having an amino acid sequence selected from the group consisting of the sequence ranging from the amino acid at position 15 to the amino acid at position 60, the sequence ranging from the amino acid at position 20 to the amino acid at position 50, the sequence ranging from the amino acid at position 24 to the amino acid at position 43 of SEQ ID NO: 1, SEQ ID NO: 2, SEQ ID NO: 3 or SEQ ID NO: 5,
at least one fragment having an amino acid sequence selected from the group consisting of the sequence ranging from the amino acid at position 100 to the amino acid at position 150, the sequence ranging from the amino acid at position 110 to the amino acid at position 140, the sequence ranging from the amino acid at position 115 to the amino acid at position 131 of SEQ ID NO: 1, SEQ ID NO: 2, SEQ ID NO: 3 or SEQ ID NO: 5, and/or
at least one fragment having an amino acid sequence selected from the group consisting of the sequence ranging from the amino acid at position 170 to the amino acid at position 211, the sequence ranging from the amino acid at position 180 to the amino acid at position 211, or the sequence ranging from the amino acid at position 190 to the amino acid at position 211 of SEQ ID NO: 1, SEQ ID NO: 2, SEQ ID NO: 3 or SEQ ID NO: 5.

According to other embodiments, the polypeptide of the invention has a sequence derived from or homologous to one of the sequences SEQ ID NO: 6, SEQ ID NO: 7 and SEQ ID NO: 8, which advantageously comprises:
at least one fragment having an amino acid sequence selected from the group consisting of the sequence ranging from the amino acid at position 10 to the amino acid at position 60, the sequence ranging from the amino acid at position 15 to the amino acid at position 50, the sequence ranging from the amino acid at position 21 to the amino acid at position 43 of SEQ ID NO: 6, SEQ ID NO: 7 or SEQ ID NO: 8,
at least one fragment having an amino acid sequence selected from the group consisting of the sequence ranging from the amino acid at position 100 to the amino acid at position 140, the sequence ranging from the amino acid at position 105 to the amino acid at position 130, the sequence ranging from the amino acid at position 112 to the amino acid at position 125 of SEQ ID NO: 6, SEQ ID NO: 7 or SEQ ID NO: 8, and
at least one fragment having an amino acid sequence selected from the group consisting of the sequence ranging from the amino acid at position 181 to the amino acid at position 217 of SEQ ID NO: 6, or the sequence ranging from the amino acid at position 172 to the amino acid at position 187 of SEQ ID NO: 7, or the sequence ranging from the amino acid at position 181 to the amino acid at position 203 of SEQ ID NO: 8.

Another aspect of the invention is the use of a nucleic acid encoding a polypeptide of the invention.

Nucleic acids of the invention, also named polynucleotides, may be DNA or RNA molecules, that encode the polypeptide defined above, while taking into account the degeneracy of the genetic code. They can be obtained by standard techniques well known by the one skilled in the art, such as in vitro DNA amplification or polymerization, in vitro gene synthesis, oligonucleotides ligation, or by a combination of these techniques.

In some embodiments, the nucleic acid has a nucleotide sequence encoding a polypeptide having an amino acid sequence selected from the group consisting of SEQ ID NO: 1, SEQ ID NO: 2, SEQ ID NO: 3, SEQ ID NO: 5, SEQ ID NO: 6, SEQ ID NO: 7 and SEQ ID NO: 8.

The Sh28GST, Sm28GST, Sb28GST, Sj28GST, Sm26GST and Sj26GST nucleic acids have sequences that are identified and listed in the databases. For instance, in the NCBI databases (https://www.ncbi.nlm.nih.gov), the nucleotide sequence of Sh28GST may be found under accession number M87799, as updated as of Apr. 9, 2020.

The nucleotide sequence of SEQ ID NO: 4 encodes the polypeptide of sequence SEQ ID NO: 1. In one embodiment, the nucleic acid has a nucleotide sequence corresponding to SEQ ID NO: 4 (see Table 2 below).

TABLE 2
Sequence of the Sh28GST nucleic acid.

| | | |
|---|---|---|
| SEQ | Schistosoma | TCTGTCTGACTGTATGATGACTGGTGATCATATCAAGGTTA |
| ID | haematobium | TCTATTTTAACGGACGCGGACGAGCTGAATCGATCCGGAT |
| NO: | Sh28GST | GACACTTGTGGCAGCTGGTGTGAACTACGAAGATGAGAGA |
| 4 | | ATTAGTTTCCAAGATTGGCCGAAAATCAAACCAACTATTCC |
| | | GGGCGGACGATTGCCTGCAGTGAAAATCACCGATAATCAT |
| | | GGGCACGTGAAATGGATGGTAGAGAGTTTGGCTATTGCAC |
| | | GGTATATGGCGAAGAAGCATCATATGATGGGAGGAACAGA |
| | | AGAGGAGTATTATAATGTTGAGAAGTTGATTGGTCAGGCT |
| | | GAAGATCTAGAACATGAATATTACAAAACTTTGATGAAGC |
| | | CAGAAGAAGAGAAACAGAAGATAATCAAAGAGATACTGA |
| | | ACGGCAAAGTACCAGTTCTTCTCGATATTATCTGCGAATCT |
| | | CTGAAAGCGTCCACAGGCAAGCTGGCTGTTGGGGATAAAG |
| | | TGACTCTAGCCGACTTAGTTCTGATTGCTGTCATTGACCAT |
| | | GTGACTGATCTGGATAAAGAATTTCTAACTGGCAAGTATCC |
| | | TGAGATCCATAAACATAGAGAAAATCTACTAGCCAGTTCA |
| | | CCGAGATTGGCGAAATATTTATCAGACAGGGCTGCAACTC |
| | | CCTTCTAGAACTGTCAACAGAATGCTGGGTGTGACGAGATT |
| | | GAAGATACTGATAGTAGTGCACTGGTGCGACCTTTTTACTA |
| | | AGACGTCATTTGTTTTATGGTATTTTTTTTCGCAATCGTTAT |
| | | TAAAATAAACTTAGTTTTCTGTTT |

In some embodiments, the nucleic acid of the invention is an isolated or purified nucleic acid.

As will be understood by those of skill in the art, it may be advantageous in some instances to produce polypeptide-encoding nucleotide molecules possessing codons non-naturally occurring in the encoded polypeptide. For example, codons preferred by a particular prokaryotic or eukaryotic host can be selected to increase the rate of recombinant polypeptide expression.

A nucleic acid according to this invention can also include sequences encoding tags, carrier proteins, signal peptides, or non-transcribed or translated sequences increasing expression or stability of the molecule.

Another aspect of the invention is the use of a vector comprising a nucleic acid encoding a polypeptide of the invention.

Preferably, the vector of the invention is an expression vector. Said expression vector comprises a nucleic acid sequence encoding a polypeptide according to the invention operatively associated with expression control elements.

Typically, the nucleic acid of the invention may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

The expression vector according to the invention may comprise a functional expression cassette. An expression cassette comprises a nucleic acid sequence encoding a polypeptide of the invention, which is operably linked to elements necessary to its expression. Said vector advantageously contains a promoter sequence, signals for initiation and termination of translation, as well as appropriate regions for regulation of translation, such as a promoter, enhancer, terminator and the like, to cause or direct expression of said polypeptide upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like.

The protein according to the invention, regardless of the schistosome from which it is derived and regardless of its molecular weight, may be natural isolated (native) or recombinant.

Throughout the present specification, the term “recombinant 28 kDa glutathione S-transferase” (rSh28GST) denotes any protein or polypeptide obtained recombinantly by inserting the complete sequence coding for Sh28GST (SEQ NO: 4) or a fraction of this sequence into a host organism. This synthesis may be carried out in various host cells, bacteria, yeasts or higher cells, as a function of the vector into which the coding sequence is inserted and the signals controlling expression. The recombinant protein according to the present invention may have a primary structure identical to that of the Sh28GST native protein, present in Schistosoma haematobium, but it may also be a derivative of the latter, or a Sh28GST incomplete protein (protein fragment), but having immunogenic activity. This protein or this protein fragment may also be fused with another protein (or protein fragment), following genetic manipulation of the corresponding DNA segments, in order to promote better expression of the protein in the host cell or optionally to cause its excretion out of the cell.

Technologies for cloning and expressing a foreign gene or a foreign gene fragment in various host cells are known to a person skilled in the art. For instance, the rSh28GST protein may be produced in Saccharomyces cerevisiae by inserting the nucleic acid of sequence SEQ NO: 4. For instance, the article “Crystal structure of the 28 kDa glutathione S-transferase from Schistosoma haematobium” teaches a method of producing the recombinant Sh28GST in Escherichia coli. Moreover, the article “Vaccine potential of a recombinant glutathione S-transferase cloned from Schistosoma haematobium in primates experimentally infected with an homologous challenge” Vaccine 1999, discloses a Sh28GST recombinant protein produced in a specific strain of Saccharomyces cerevisiae.

In one embodiment, the polypeptide according to the invention is the expression product of the nucleic acid of sequence SEQ ID NO: 4 in Saccharomyces cerevisiae or in Escherichia coli.

The recombinant protein of the invention may also be produced according to other methods, or in other host cells, which are well known in the art.

It is also possible to choose to use gene therapy, by using or administering a nucleic acid coding for a polypeptide of the invention instead of the polypeptide. In this case, it is administered to the patient a nucleic acid encoding the polypeptide of interest under conditions such that the polypeptide is expressed in vivo by the patient's cells into which the nucleic acid has been transferred.

The invention therefore also concerns nucleic acids comprising or consisting of a sequence encoding a polypeptide of the invention. Said nucleic acids may easily be obtained by cloning fragments of cDNA coding for a polypeptide of the invention.

Such a nucleic acid coding for a polypeptide of the invention may particularly be in the form of a DNA vector, for example a plasmid vector. It is possible to administer one or more vectors, each vector possibly carrying one or more sequences coding for at least one of the polypeptides of the invention. In this vector, the sequence(s) coding for at least one of the polypeptides of the invention are functionally linked to an element or elements allowing expression thereof or regulation of the expression thereof such as transcriptional promoters, activators and/or terminators.

According to one preferred embodiment, a vector is used carrying a sequence coding for a polypeptide of the invention.

The DNA vector or vectors may be inserted in vivo using any technique known to persons skilled in the art. In particular, it is possible to insert the DNA vector or vectors in vivo in naked form i.e. without the assistance of any vehicle or system which would facilitate transfection of the vector in the cells (EP 465 529).

A gene gun can also be used, for example by depositing DNA on the surface of “gold” particles and shooting these particles so that the DNA passes through a patient's skin (Tang et al., (1992) Nature 356:152-4). Injections using a liquid gel are also possible to transfect skin, muscle, fat tissue and mammary tissue all at the same time (Furth et al., (1992) Anal Biochem. 205:365-8).

Other available techniques include micro-injection, electroporation, precipitation with calcium phosphate, formulations using nanocapsules or liposomes.

Biodegradable nanoparticles in polyalkyl cyanoacrylate are particularly advantageous. For liposomes, the use of cationic lipids promotes the encapsulation of negatively-charged nucleic acids and facilitates fusion with the negatively-charged cell membranes.

Alternatively, the vector may be in the form of a recombinant virus which, inserted in its genome, comprises a nucleic acid sequence coding for the said polypeptide(s).

The viral vector may preferably be selected from an adenovirus, a retrovirus, in particular a lentivirus, and an adeno-associated virus (AAV), a herpes virus, a cytomegalovirus (CMV), a vaccine virus, etc. Lentivirus vectors are described for example by Firat et al., (2002) J Gene Med 4:38-45.

Advantageously, the recombinant virus is a defective virus. The term “defective virus” denotes a virus incapable of replicating in a target cell. In general, the genome of defective viruses is devoid of at least the sequences needed for replication of the said virus in the infected cell. These regions can either be eliminated or made non-functional or can be substituted by other sequences and in particular by the nucleic acid which encodes the polypeptide of interest. Nonetheless, preferably the defective virus maintains the sequences of its genome which are needed for encapsulating the viral particles.

Such defective viruses may be produced by techniques known in the art, such as by transfecting packaging cells or by transient transfection with helper plasmids or viruses.

The targeted administration of genes is described for example in application WO 95/28 494.

A further object of the present invention is the use of a composition comprising a polypeptide according to the present invention.

A further object of the present invention is the use of a composition comprising a nucleic acid encoding a polypeptide according to the present invention.

A further object of the present invention is the use of a composition comprising an expression vector comprising a nucleic acid encoding a polypeptide according to the present invention.

A further object of the present invention is the use of a pharmaceutical composition comprising a polypeptide according to the present invention and at least one pharmaceutically acceptable excipient.

A further object of the present invention is the use of a pharmaceutical composition comprising a nucleic acid encoding a polypeptide according to the present invention and at least one pharmaceutically acceptable excipient.

A further object of the present invention is the use of a pharmaceutical composition comprising an expression vector comprising a nucleic acid encoding a polypeptide according to the present invention and at least one pharmaceutically acceptable excipient or vehicle.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene- block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

A further object of the present invention is the use of a medicament comprising a polypeptide according to the present invention.

A further object of the present invention is the use of a medicament comprising a nucleic acid encoding a polypeptide according to the present invention.

A further object of the present invention is the use of a medicament comprising an expression vector comprising a nucleic acid encoding a polypeptide according to the present invention.

In one embodiment, the composition, pharmaceutical composition of the invention is a vaccine composition. In one embodiment, the vaccine composition of the invention comprises at least one adjuvant.

In one embodiment, the composition, pharmaceutical composition, or vaccine of the invention thus comprise one or more adjuvants.

Suitable adjuvants that may be used in the present invention include, but are not limited to:
(1) natural or non-natural aluminum salts (alum), such as, for example, aluminum hydroxide, aluminum phosphate, aluminum sulfate (hydrated or not), alum (KAl(SO4)2.12H2O), any other salt of formula (BAl(SO4)2.12H2O), etc.;
(2) oil-in-water emulsion formulations (with or without other specific immunostimulating agents such as, for example, muramyl peptides (defined below) or bacterial cell wall components), such as, for example, squalene-based emulsions (e.g., squalene-based oil-in-water emulsions) or squalane-based emulsions, such as, for example,
(a) MF59 (a squalene-based oil-in-water adjuvant described in WO 90/14837), containing 5% squalene, 0.5% Tween 80, and 0.5% span 85 (optionally containing various amounts of MTP-PE (see below, although not required)) formulated into submicron particles using a microfluidizer such as Model 110Y microfluidizer (Microfluidics, Newton, Mass.),
(b) SAF, containing 10% Squalene, 0.4% Tween 80, 5% pluronic-blocked polymer L121, and thr-MDP (see below) either microfluidized into a submicron emulsion or vortexed to generate a larger particle size emulsion, and
(c) Ribi™ adjuvant system (RAS), (Corixa, Hamilton, Mont.) containing 2% squalene, 0.2% Tween 80, and one or more bacterial cell wall components from the group consisting of 3-O-deaylated monophosphorylipid A (MPL™) described in U.S. Pat. No. 4,912,094 (Corixa), trehalose dimycolate (TDM), and cell wall skeleton (CWS), preferably MPL+CWS (Detox™);
(d) squalane based adjuvant comprising but not limited to the following composition: squalane 3.9%, w/v, sorbitan trioleate (0.47%, w/v), and polyoxyethylene (80) sorbitan monooleate (0.47%, w/v) dispersed in citrate buffer;
(3) water-in-oil emulsion formulations, such as, for example, ISA-51 or squalene-based water-in-oil adjuvant (e.g., ISA-720); Oil adjuvants suitable for use in water-in-oil emulsions may include mineral oils and/or metabolizable oils. Mineral oils may be selected from Bayol®, Marcol.®. and Drakeol, including Drakeol® 6VR (SEPPIC, France).®. Metabolisable oils may be selected from SP oil (hereinafter described), Emulsigen (MPV Laboratories, Ralston, NZ), Montanide 264,266,26 (Seppic SA, Paris, France), as well as vegetable oils, animal oils such as the fish oils squalane and squalene, and tocopherol and its derivatives.
(4) saponin adjuvants, such as Quil A or STIMULON™ QS-21 (Antigenics, Framingham, Mass.) (U.S. Pat. No. 5,057,540) may be used or particles generated therefrom such as ISCOMs (immunostimulating complexes);
(5) bacterial lipopolysaccharides, synthetic lipidA analogs such as aminoalkyl glucosamine phosphate compounds (AGP), or derivatives or analogs thereof, which are available from Corixa, and which are described in U.S. Pat. No. 6,113,918; one such AGP is 2-[(R)-3-Tetradecanoyloxytetradecanoylamino]ethyl 2-Deoxy-4-O-phosphono-3-Oi[(R)-3tetradecanoyloxytetradecanoyl]-2-[(R)-3-tetradecanoyloxytetradecanoyl amino]-b-Dglucopyranoside, which is also known as 529 (formerly known as RC529), which is formulated as an aqueous form or as a stable emulsion, synthetic polynudeotides such as oligonucleotides containing CpG motif(s) (U.S. Pat. No. 6,207,646);
(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.;
(7) detoxified mutants of a bacterial ADP-ribosylating toxin such as a cholera toxin (CT) either in a wild-type or mutant form, for example, where the glutamic acid at amino acid position 29 is replaced by another amino acid, preferably a histidine, in accordance with published international patent application number WO 00/18434 (see also WO 02/098368 and WO 02/098369), a pertussis toxin (PT), or an E. coli heat-labile toxin (LT), particularly LT-K63, LT-R72, CT-S109, PT-K9/G129 (see, e.g., WO 93/13302 and WO92/19265); and
(8) other substances that act as immunostimulating agents to enhance the effectiveness of the composition. Muramyl peptides include, but are not limited to, N-acetylmuramyl-L-threonyl-D-isoglutamine (thr-MDP), N-acetylnormuramyl-L-alanine-2-(1′2′dipalmitoyl-sn-glycero-3hydroxyphosphoryloxy)-ethylamine (MTP-PE), etc.

In some embodiments, the adjuvant is an aluminum salt, and more particularly aluminum hydroxide.

The adjuvant used may depend, in part, on the recipient organism. Moreover, the amount of adjuvant to administer will depend on the type and size of animal.

The concentration of adjuvant may for instance be comprised from 0.5 mg/ml to 2 mg/ml, in particular from 0.3 mg/ml to 1 mg/ml and in particular from 220 μg/ml to 280 μg/ml and preferably approximately equal to 250 μg/ml.

In one embodiment, the adjuvant is aluminum hydroxide used at a concentration comprised within the range of concentrations mentioned above and in particular at 250 μg/ml.

A further object of the present invention is the use of a combination, pharmaceutical combination, or kit-of-parts comprising a polypeptide according to the present invention and at least one adjuvant as described hereabove.

The administration of each part of the combination, pharmaceutical combination, or kit-of-parts can be done simultaneously, separately or sequentially.

A first aspect of the invention is a method of preventing or treating vasculitis in a subject in need thereof, comprising administering to the subject a therapeutically effective amount of a polypeptide, a nucleic acid, a vector, a composition, a pharmaceutical composition, or a vaccine composition of the invention.

Another aspect is a method of preventing or treating atherosclerosis, endometriosis, hypertension, osteonecrosis, Parkinson's disease, steatohepatitis, obesity-induced pathologies, lipodystrophy, or myocardial infarction in a subject in need thereof, comprising administering to the subject a therapeutically effective amount of a polypeptide, a nucleic acid, a vector, a composition, a pharmaceutical composition, or a vaccine composition of the invention.

Another aspect is a polypeptide, a nucleic acid, a vector, a composition, a pharmaceutical composition, or a vaccine composition of the invention, for use in the preventive or therapeutic treatment of vasculitis.

Another aspect is a polypeptide, a nucleic acid, a vector, a composition, a pharmaceutical composition, or a vaccine composition of the invention, for use in the preventive or therapeutic treatment of atherosclerosis, endometriosis, hypertension, osteonecrosis, Parkinson's disease, steatohepatitis, obesity-induced pathologies, lipodystrophy, or myocardial infarction.

A further aspect is the use of a polypeptide, a nucleic acid, a vector, a composition, a pharmaceutical composition, or a vaccine composition of the invention, for the manufacture of a medicament for the preventive or therapeutic treatment of vasculitis.

A further aspect is the use of a polypeptide, a nucleic acid, a vector, a composition, a pharmaceutical composition, or a vaccine composition of the invention, for the manufacture of a medicament for the preventive or therapeutic treatment of atherosclerosis, endometriosis, hypertension, osteonecrosis, Parkinson's disease, steatohepatitis, obesity-induced pathologies, lipodystrophy, or myocardial infarction.

Another aspect is a polypeptide, a nucleic acid, a vector, a composition, or a pharmaceutical composition of the invention, for use as a vaccine in the preventive or therapeutic treatment of vasculitis.

Another aspect is a polypeptide, a nucleic acid, a vector, a composition, or a pharmaceutical composition of the invention, for use as a vaccine in the preventive or therapeutic treatment of atherosclerosis, endometriosis, hypertension, osteonecrosis, Parkinson's disease, steatohepatitis, obesity-induced pathologies, lipodystrophy, or myocardial infarction.

A further aspect is the use of a polypeptide, a nucleic acid, a vector, a composition, or a pharmaceutical composition of the invention, for the manufacture of a vaccine for the preventive or therapeutic treatment of vasculitis.

A further aspect is the use of a polypeptide, a nucleic acid, a vector, a composition, or a pharmaceutical composition of the invention, for the manufacture of a vaccine for the preventive or therapeutic treatment of atherosclerosis, endometriosis, hypertension, osteonecrosis, Parkinson's disease, steatohepatitis, obesity-induced pathologies, lipodystrophy, or myocardial infarction.

The polypeptide, a nucleic acid, a vector, a composition, a pharmaceutical composition, or a vaccine composition of the invention are suitable and useful for the preventive or therapeutic treatment of a disease characterized by a M1/M2 macrophage ratio dysregulation. Such M1/M2 macrophage ratio dysregulation may be due to a decrease of the M1-type immune response and/or an increase of the M2-type immune response. Various diseases have been shown to involve or be associated with M1/M2 macrophage ratio dysregulation. Non-limiting examples of such diseases associated with M1/M2 macrophage ratio dysregulation include e.g. atherosclerosis, endometriosis, hypertension, osteonecrosis, Parkinson's disease, steatohepatitis, obesity-induced pathologies, lipodystrophy and myocardial infarction.

Therefore, the polypeptide, nucleic acid, vector, composition, pharmaceutical composition, or vaccine composition of the invention are suitable and useful for the preventive or therapeutic treatment of a disease selected from the group consisting of atherosclerosis, endometriosis, hypertension, osteonecrosis, Parkinson's disease, steatohepatitis, obesity-induced pathologies, lipodystrophy and myocardial infarction.

The polypeptide, nucleic acid, vector, composition, pharmaceutical composition, or vaccine composition of the invention are also suitable and useful for the preventive or therapeutic treatment of any type of vasculitis.

According to the International Chapel Hill Consensus Conference on the Nomenclature of Vasculitides (CHCC 2012), vasculitis can be classified according to:
a) the size of vessels involved, which may be:
   large vessels (e.g. aorta, coronary arteries)
   medium vessels (e.g. medium or small arteries)
   small vessels (e.g. Antineutrophil cytoplasmic antibody (ANCA)-associated vasculitis, immune complex)
   variable vessels (Behçet's disease, Cogan disease)
b) the organs or tissues involved, which may be:
   multi-organs (Behçet's disease, immune complex)
   single organs (skin, testicular, central nervous system . . . )
c) association with other diseases (e.g. lupus, rheumatoid arthritis, syphilis, cancer . . . ).

In the context of the invention, the type of vasculitis to be prevented or treated may be any kind of vasculitis, such as e.g.:
variable vessel vasculitis (VVV) including Behçet's disease (BD) and Cogan's syndrome (CS);
large vessel vasculitis (LVV) including Takayasu arteritis (TAK) and Giant cell arteritis (GCA);
medium vessel vasculitis (MVV) including Polyarteritis nodosa (PAN) and Kawasaki disease (KD);
small vessel vasculitis (SVV) including Antineutrophil cytoplasmic antibody (ANCA)-associated vasculitis (AAV) (such as Microscopic polyangiitis (MPA), Granulomatosis with polyangiitis (Wegener's) (GPA), Eosinophilic granulomatosis with polyangiitis (Churg-Strauss) (EGPA)) and Immune complex SVV (such as Anti-glomerular basement membrane (anti-GBM) disease, Cryoglobulinemic vasculitis (CV), IgA vasculitis (Henoch-Schanlein) (IgAV), Hypocomplementemic urticarial vasculitis (HUV) (anti-C1q vasculitis));
single-organ vasculitis (SOV) including Cutaneous leukocytoclastic angiitis, Cutaneous arteritis, Primary central nervous system vasculitis, Isolated aortitis;
vasculitis associated with systemic disease including Lupus vasculitis, Rheumatoid vasculitis, Sarcoid vasculitis; or
vasculitis associated with probable etiology including Hepatitis C virus-associated cryoglobulinemic vasculitis, Hepatitis B virus-associated vasculitis, Syphilis-associated aortitis, Drug-associated immune complex vasculitis, Drug-associated ANCA-associated vasculitis, and Cancer-associated vasculitis.

Therefore, in some embodiments, vasculitis is selected from the group consisting of Behçet's disease (BD), Cogan's syndrome (CS), Takayasu arteritis (TAK), Giant cell arteritis (GCA), Polyarteritis nodosa (PAN), Kawasaki disease (KD), Antineutrophil cytoplasmic antibody (ANCA)-associated vasculitis (AAV), Microscopic polyangiitis (MPA), Granulomatosis with polyangiitis (Wegener's) (GPA), Eosinophilic granulomatosis with polyangiitis (Churg-Strauss) (EGPA)), Immune complex small vessel vasculitis, Anti-glomerular basement membrane (anti-GBM) disease, Cryoglobulinemic vasculitis (CV), IgA vasculitis (Henoch-Schanlein) (IgAV), Hypocomplementemic urticarial vasculitis (HUV) (anti-C1q vasculitis), Cutaneous leukocytoclastic angiitis, Cutaneous arteritis, Primary central nervous system vasculitis, Isolated aortitis.

In one embodiment, vasculitis to be prevented or treated is Behçet's disease (BD).

In an embodiment, said vasculitis is associated to another disease selected from the group consisting of Lupus, Rheumatoid arthritis, Sarcoidosis, Hepatitis C, Hepatitis B, Syphilis and Cancer.

In one embodiment, the polypeptide, nucleic acid, vector, composition, pharmaceutical composition or vaccine composition according to the present invention will be formulated for administration to the subject.

The method of administration of the polypeptide, nucleic acid, vector, composition, pharmaceutical composition or vaccine composition of the invention is not limited.

In one embodiment, the polypeptide, nucleic acid, expression vector, composition, pharmaceutical composition or medicament according to the present invention is to be administered systemically or locally.

In one embodiment, the polypeptide, nucleic acid, expression vector, composition, pharmaceutical composition or medicament according to the present invention is to be administered by injection (for instance by subcutaneous injection), rectally, orally, topically, nasally, buccally, vaginally, intratracheally, by endoscopy, transmucosally, or by percutaneous administration.

In one embodiment, the polypeptide, nucleic acid, expression vector, composition, pharmaceutical composition or medicament according to the present invention is to be injected, preferably systemically injected.

Examples of systemic injections include, but are not limited to, intravenous (iv), subcutaneous, intramuscular (im), intradermal (id), intraperitoneal (ip) injection and perfusion.

In one embodiment, the polypeptide, nucleic acid, expression vector, composition, pharmaceutical composition or medicament according to the present invention is to be subcutaneously injected.

In one embodiment, the polypeptide, nucleic acid, expression vector, composition, pharmaceutical composition or medicament according to the present invention is to be administered intrarectally.

The polypeptide, nucleic acid, expression vector, composition, pharmaceutical composition or medicament according to the present may be administered, for example, in the form of a nasal or buccal spray, a suppository, a tablet, a lyophilizate, a capsule, a syrup, a solution injectable by the intravenous, subcutaneous or intramuscular route, an ointment or gel for topical application.

Examples of formulations adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

In one embodiment, when injected, the polypeptide, nucleic acid, expression vector, composition, pharmaceutical composition or medicament according to the present invention is sterile. Methods for obtaining a sterile composition include, but are not limited to, GMP synthesis (where GMP stands for “Good manufacturing practice”).

Sterile injectable forms of a composition may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

It will be understood that other suitable routes of administration are also contemplated in the present invention, and the administration mode will ultimately be decided by the attending physician within the scope of sound medical judgment. Apart from administration by injection, other routes are available, such as e.g. nebulization.

In one embodiment, the polypeptide, nucleic acid, expression vector, composition, pharmaceutical composition or medicament according to the present invention is to be administered to the subject in need thereof in a therapeutically effective amount.

The term “therapeutically effective amount”, as used herein, refers to an amount effective, at dosages and for periods of time necessary, to achieve a desired preventive and/or therapeutic result.

It will be however understood that the total daily usage of the polypeptide, nucleic acid, expression vector, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the polypeptide, nucleic acid, expression vector, composition, pharmaceutical composition or medicament employed; the age, body weight, general health, sex and diet of the subject; the time of administration, route of administration, and rate of excretion of the specific isolated antibody or binding fragment thereof, nucleic acid, expression vector, composition, pharmaceutical composition or medicament employed; the duration of the treatment; drugs used in combination or coincidental with the specific polypeptide, nucleic acid, expression vector, composition, pharmaceutical composition or medicament employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, a therapeutically effective amount of the isolated polypeptide, administered alone or within a composition, pharmaceutical composition or medicament according to the present invention ranges from about 10 μg/kg to about 2000 μg/kg, from about 20 μg/kg to about 1750 μg/kg, from about 30 μg/kg to about 1500 μg/kg, from about 40 μg/kg to about 1250 μg/kg, from about 50 μg/kg to about 1000 μg/kg.

For instance, the therapeutically effective dose may be equal to 50 μg/kg, 500 μg/kg or 1000 μg/kg.

The amount of purified protein administered per subject may be, for example, equal to 253 μg (which may correspond to a final dose of 100 μg of vaccine comprising rSh28GST28 plus aluminum hydroxide at a concentration of 1 mg/ml). The dose of protein may be, for example, greater than or equal to 100 μg and less than or equal to 500 μg of protein.

In one embodiment, a therapeutically effective amount of the polypeptide, nucleic acid, expression vector, composition, pharmaceutical composition or medicament according to the present invention is to be administered once a day, twice a day, three times a day or more.

In one embodiment, a therapeutically effective amount of the polypeptide, nucleic acid, expression vector, composition, pharmaceutical composition or medicament according to the present invention is to be administered every day, every two days, every three days, every four days, every five days, every six days.

In one embodiment, a therapeutically effective amount of the polypeptide, nucleic acid, expression vector, composition, pharmaceutical composition or medicament according to the present invention is to be administered every week, every two weeks, every three weeks.

In one embodiment, a therapeutically effective amount of the polypeptide, nucleic acid, expression vector, composition, pharmaceutical composition or medicament according to the present invention is to be administered every month, every two months, every three months, every four months, every five months, every six months.

In a preferred embodiment, a therapeutically effective amount of the polypeptide, nucleic acid, expression vector, composition, pharmaceutical composition or medicament according to the present invention is to be administered every 12 hours, every 24 hours, every 36 hours, every 48 hours, every 60 hours, every 72 hours, every 96 hours.

In a preferred embodiment, a therapeutically effective amount of the polypeptide, nucleic acid, expression vector, composition, pharmaceutical composition or medicament according to the present invention is to be administered every 60 hours.

In one embodiment, the polypeptide, nucleic acid, expression vector, composition, pharmaceutical composition or medicament according to the present invention is for acute administration. In one embodiment, the polypeptide, nucleic acid, expression vector, composition, pharmaceutical composition or medicament according to the present invention is for chronic administration.

In one embodiment, a therapeutically effective amount of the polypeptide, nucleic acid, expression vector, composition, pharmaceutical composition or medicament according to the present invention is to be administered for about 5 days, 7 days, 10 days, 14 days, 21 days, 28 days, 1 month, 2 months, 3 months, 6 months, 1 year or more.

In one embodiment, a therapeutically effective amount of the polypeptide, nucleic acid, expression vector, composition, pharmaceutical composition or medicament according to the present invention is to be administered for a period of time ranging from about one week to about eight weeks, from about two weeks to about seven weeks, from about two weeks to about six weeks, from about two weeks to about five weeks.

In a preferred embodiment, a therapeutically effective amount of the polypeptide, nucleic acid, expression vector, composition, pharmaceutical composition or medicament according to the present invention is to be administered for a period of time ranging from about 10 days to about 40 days, from about 15 days to about 35 days, from about 20 days to about 30 days.

In one embodiment, the polypeptide of the invention is used in combination with at least one adjuvant. Examples of adjuvants are described hereabove in the specification.

The administration of the polypeptide of the invention and the at least one adjuvant may be simultaneous, separate or sequential. For simultaneous administration the agents may be administered as one composition or as separate compositions, as appropriate.

The polypeptide of the invention may be administered before, concomitantly with, or after, the at least one adjuvant.

### BRIEF DESCRIPTION OF THE DRAWINGS

FIG. 1 is a diagram of the protocol used for studying the prophylactic effect of P28GST in a skin inflammation mouse model. Mice were immunized with 3 subcutaneous injections of P28GST (0.5 μg/kg) with an adjuvant (Alum), prior to imiquimod-induction of skin inflammation. Different negative controls were used: mice with only NaCl injections (Control), mice with NaCl injections and imiquimod application (IMQ) and mice with injections of adjuvant alone and then application of imiquimod (Placebo). Betamethasone, an anti-inflammatory treatment, was used as a reference treatment.

FIG. 2 represents the clinical score obtained after imiquimod-induction of skin inflammation in mice immunized with P28GST before imiquimod application, measured daily during the 5 days of imiquimod application. Different negative controls were used: mice with only NaCl injections (Control), mice with NaCl injections and imiquimod application (IMQ) and mice with injections of adjuvant alone and then application of imiquimod (Placebo). Betamethasone, an anti-inflammatory treatment, was used as a reference treatment. *p<0.05 (P28 vs Placebo) and ##p<0.01 (P28 vs IMQ).

FIG. 3 represents the relative mRNA expression of the inflammation markers TNFα and IL-1β (normalized with the β-actin gene) in mice immunized with P28GST before imiquimod application. Different negative controls were used: mice with only NaCl injections (Control), mice with NaCl injections and imiquimod application (IMQ) and mice with injections of adjuvant alone and then application of imiquimod (Placebo).

FIG. 4 represents the percentage of CD80+ macrophages and of CD206+ macrophages in the F4/80+ spleen population, the M2/M1 ratio in the spleen, as indicated by the F4/80+CD80+ cell to F4/80+CD206+ cell ratio, and in the skin, as indicated by the Arginase/iNOS mRNA expression ratio, after immunization with P28GST and imiquimod application. Pro-inflammatory M1 macrophages are CD80+ and iNOS+ and anti-inflammatory M2 macrophages are CD206+ and Arginase+. Different negative controls were used: mice with only NaCl injections (Control), mice with NaCl injections and imiquimod application (IMQ) and mice with injections of adjuvant alone and then application of imiquimod (Placebo). *p<0.05, **p<0.01 and ***p<0.001.

FIG. 5 is a diagram of the study design. Vasculitis induction was performed during the first three days and P28GST (5 μg/kg) injected with an adjuvant (Alum), or anti-TNFα (300 μg/rat), were administered to Lewis rats at day 18, 25 and 32. Different negative controls were used: mice with vasculitis induction and NaCl injection (Nacl), mice with vasculitis induction and injection of adjuvant alone (Placebo). Anti-TNFα, an anti-inflammatory treatment, was used as a positive control.

FIG. 6 represents histograms showing the concentration of Nitrite (A), Urea (B), Lipocalin-2 (C) and Timp-1 (D) at 25 days (A and C) and 32 days (B and D) in rats treated with control (NaCl), placebo (adjuvant) or P28GST (at 5 μg/kg, with adjuvant). Statistical analyses were performed using a Mann Whitney test and were represented as follows: *p<0.05 and **p<0.01.

FIG. 7 represents histograms showing the concentration of Nitrite (A), Urea (B), Lipocalin-2 (C) and Timp-1 (D) at 25 days (A and C) and 32 days (B and D) in rats treated with control (NaCl), P28GST (at 5 μg/kg, with adjuvant) or anti-TNFα. Statistical analyses were performed using a Mann Whitney test and were represented as follows: *p<0.05 and **p<0.01.

### EXAMPLES

The present invention is further illustrated by the following examples.

### Example 1

Materials and Methods

Animals and Ethical Considerations

Four-week-old male BALB/C mice were purchased from Janvier Labs (Le Genest-Saint-Isle, France). The mice were maintained in pathogen-free animal holding facilities, controlled for clinical and behavioral signs of pain, and weighed daily. All experiments were approved by the local animal ethics committee and the Ministry of Higher Education, Research, and Innovation.

Immunizations and Induction of Skin Inflammation

Mice were immunized every 2 weeks with 3 subcutaneous injections of P28GST at 0.5 μg/kg. A week after the last injection of P28GST skin inflammation was induced by daily application of 62.5 mg imiquimod (Aldara®, 5%, MEDA Pharma S.A.) on shaved abdominal skin for 5 consecutive days as described previously (van der Fits et al., 2009). One group was treated with 50 mg betamethasone (Betneval 0.1% cream) directly on the injured skin, 5 hours after IMQ application. Mice were sacrificed by lethal anesthesia using pentobarbital (Dolethal®, Vetoquinol). Skin lesions were excised for histological analysis and real-time quantitative PCR analysis.

Chemicals and Reagents

Recombinant ShP28GST protein was expressed in cultured Saccharomyces cerevisiae and purified under Good Manufacturing Practice conditions by Eurogentec S.A (Seraing, Belgium). Batches of P28GST (batchM-BIX-P03-225a) were conserved by lyophilizing in 10 mM NH4HCO3 and 2.8% lactose. This preparation was re-suspended extemporaneously using 0.9% NaCl (Aguettant, Lyon, France) or 0.2% alhydrogel (Eurogentec S.A., Seraing, Belgium) at the appropriate concentrations.

ARN Extraction and RT-qPCR

Total RNA was extracted from skin using a nucleospin RNA kit (Macherey Nagel, Hoerdt, France) after being lysed with TRIzol (Thermo Fisher Scientific, Waltham, Mass.) in a Precellys homogenizer. The RNA concentration and purity were determined by the absorbance at 260 nm and 280 nm using a NanoDrop 1000 (Thermo Fisher Scientific, Waltham, Mass.). Retro-transcription was performed using the Superscript RT kit (Applied Biosystems, Foster City, Calif.) and 1 μg of RNA. Gene expression was evaluated by RT-qPCR using Fast SYBR Green Master Mix reagent on a StepOne instrument (Applied Biosystems, Foster City, Calif.) and normalized to β-actin. The following mice primers were used for IL-1β forward AGCTCTCCACCTCAATGGAC (SEQ ID NO: 9) and reverse AGGCCACAGGTATTTTGTCG (SEQ ID NO: 10), TNFα forward CCTGTAGCCCACGTCGTAG (SEQ ID NO: 11) and reverse GGGAGTAGACAAGGTACAACCC (SEQ ID NO: 12), β-actin forward CCTTCTTGGGTATGGAATCCT (SEQ ID NO: 13) and reverse CTTTACGGATGTCAACGTCAC (SEQ ID NO: 14), iNOS forward CAGCTGGGCTGTACAAACCTT (SEQ ID NO: 15) and reverse CATTGGAAGTGAAGCGTTTCA (SEQ ID NO: 16), Arg1 forward CAGAAGAATGGAAGAGTCAG (SEQ ID NO: 17) and reverse CAGATATGCAGGGAGTCACC (SEQ ID NO: 18). Results were expressed as relative expression compared to control using the 2-ΔΔCt method.

Clinical Score

The severity of inflammation was blindly evaluated each day using the Psoriasis Area and Severity Index (PASI) adapted to mice according to van der Fits. Three parameters (erythema, scaling and thickening) were scored from 0 to 4 (0: absent, 1: slightly, 2: moderate, 3: marked and 4: severe). The cumulative score (from 0 to 12) was used to evaluate skin inflammation.

Flow Cytometry

Spleen cells were extracted after sacrifice and maintained in culture medium after 70-μm filtration and red blood cell lysis. Cells were blocked in 2.4G2 (BD Bioscience) and viability assessed using fixable viability dye (eBioscience, Thermo Fisher Scientific, Waltham, Mass.). Cell surface immunostaining was performed, followed by intra-nuclear immunostaining after fixation and permeation using the true-nuclear factor kit (Ozyme, Saint-Cyr, France). Cells were analyzed on a Fortessa X20 (BD Biosciences, San Jose, Calif.) using the following antibodies fixable-viability dye (13539140), F4/80 (12-4801), CD206 (141729) and CD80 (15-0801) from eBiosciences and Biolegend. Data analysis was performed using FlowJo Software (Tree star, Ashland, Oreg.).

Statistical Analysis

Clinical scores were presented as mean±standard error of the mean and analyzed by two-way ANOVA+Bonferroni post-test. All other results were presented as medians. The Mann Whitney non parametric t-test was used to compare two groups and the Kruskal-Wallis+Dunn's post-test when more than two groups were compared. Each test was performed using an alpha level of 0.5%, and results were considered significant when p<0.05. Statistical analyses were performed in GraphPad Prism 5 (GraphPad Software, La Jolla, Calif.).

Results

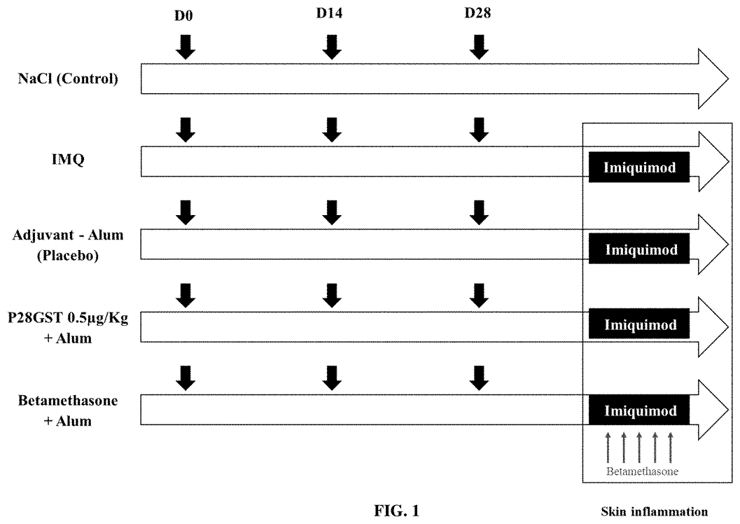

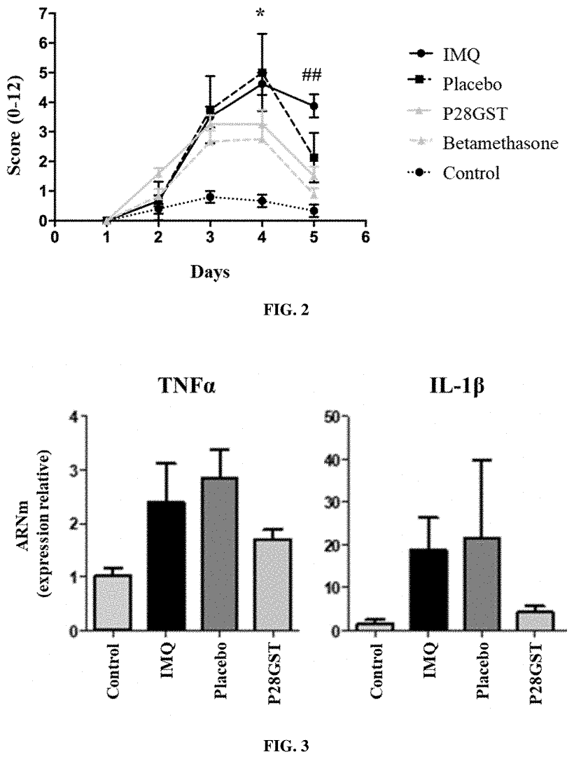

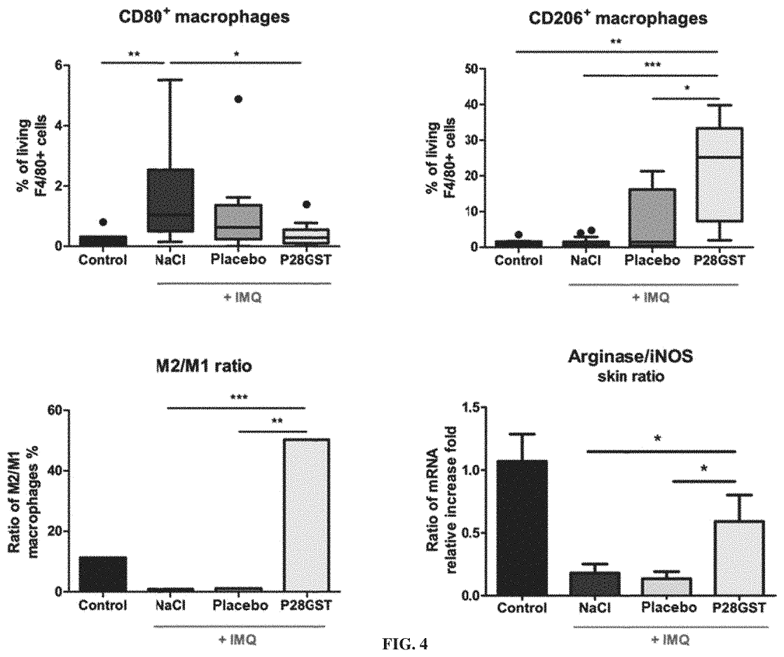

The goal was to test the immunization process with P28GST in an experimental imiquimod-induced skin inflammation Balb/C mice model. Vasculitis being a systemic disease characterized by typical skin inflammation, this animal model is a suitable model of vasculitis. Mice received 3 subcutaneous injections of P28GST (0.5 μg/kg) with adjuvant (Alum) at days 1, 14 and 28. Then, imiquimod was applied into the skin of immunized mice daily during 5 days (FIG. 1). P28GST immunization reduced erythema, as shown by the Clinical score (FIG. 2). P28GST immunization also led to a decrease of the relative expression of the pro-inflammatory cytokines TNFα and IL-1β (FIG. 3). Interestingly, this was associated with a decrease of M1 pro-inflammatory macrophages (CD80+ and iNOS+) and an increase of M2 anti-inflammatory macrophages (CD206+ and Arginase+) in the spleen as well as an increase of the M2/M1 ratio in the skin (FIG. 4). These results showed the induction of M2 macrophages as well as a polarization towards a M2-type immune response. P28GST can thus modulate the inflammatory response in order to reduce skin inflammation, representing a new approach of prevention or treatment of patients suffering from a disease characterized by a M1/M2 macrophage ratio dysregulation, and patients with vasculitis including patients with Behçet's disease (BD).

### Example 2

Materials and Methods

Vasculitis Induction

Lewis rats were immunized as described below, on Day 1, the Behçet-like disease (BD) was induced in the rats of the TPM-induced BD groups by injection in both hind foot pads with TPM in the presence of CFA (50 μg/rat; i.e. 25 μg in 50 μL per hind foot pad). The same day and on Day 3, the rats were intraperitoneally (IP) injected with 200 ng of Pertussis Toxin.

On Day 14, a bioluminescence acquisition was performed on rats in order to assess the development of the BD. As the TPM-induced BD model was not considered as confirmed, another bioluminescence acquisition was performed on Day 17. This last bioluminescence acquisition showed a sufficient systemic inflammation to begin the treatment test. Then, on Day 18, rats were treated with P28GST test item, adjuvant alone, anti-rat TNF-α or saline solution.

Study Design

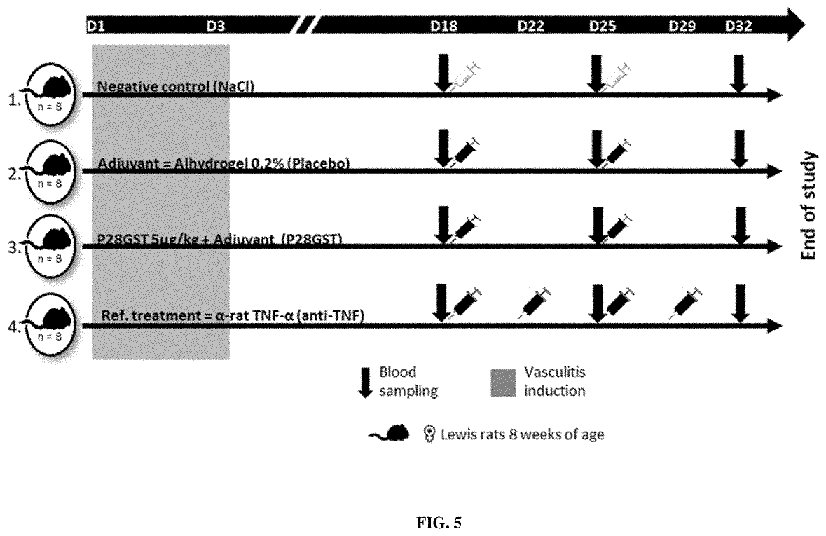

Four groups were tested to evaluate the effect of P28GST (5 μg/kg) therapeutic treatment on symptoms evolution and tissues inflammation, when administered subcutaneously after systemic inflammatory disease induction in rodents. In the study design, different negative controls were used: mice with vasculitis induction and NaCl injection (Nacl), mice with vasculitis induction and injection of adjuvant (Placebo), two doses were injected at day 18 and day 25. Anti-TNFα, a reference drug in the treatment of various inflammatory-mediated autoimmune diseases was used as a positive control (FIG. 5). For anti-TNF treatment, four doses of 300 μg/rat were injected intraperitoneally at day 18, day 22, day 25 and day 29. P28GST (5 μg/kg) was injected at day 18 and day 25.

Samples

Whole blood (WB) was sampled once a week in order to assess the level of nitrite and urea and some systemic cytokines. About 1 mL per rat was sampled and deposited in Lithium/Heparin tubes. Tubes were mixed gently in order to ensure an optimal homogenization between blood and anticoagulant and then a centrifugation was performed at 2000 g for 5 minutes at room temperature. The supernatant (i.e. plasma) was split into two microcentrifuge tubes. One tube was stored at −20° C. until biochemical analysis (Nitrite and Urea measurement) and the other tube was stored at −80° C. until Cytokine analysis.

After euthanasia, eyes were harvested for TIMP-1 analysis. Eyes were sampled and then crushed in Reagent Diluent Concentrate 2 for protein extraction. The dosage of total proteins was performed with Pierce Coomassie assay kit (Thermo Fisher Scientific, Waltham, Mass.). Then, protein extracts were stored at −80° C. until Timp-1 ELISA analysis.

Biochemical Analysis

Nitrite: Nitrite concentration was measured by photometric (Griess Reagent) determination at 540 nm using the reagent Griess Reagent Kit for Nitrite Determination G-7921 provided by Thermo Fisher. The measuring range was 1-100 μM.

Urea (UREE): Urea concentration was measured by photometric determination at 520 nm using the reagent Urea 981820 provided by Thermo Fisher Diagnostics. The measuring range was 1.5-75.0 mmol/L, the detection limit (zero sample+3 SD) was 1.1 mmol/L and the within-run and between-run imprecisions range between 1.9% and 6.4%.

Lipocalin-2: The test kit was a solid phase enzyme immunometric assay (ELISA) in the microplate format, designed for the quantitative measurement of Rat Lipocalin-2.

The microplate was coated with a capture antibody. Then, calibrators and samples were added for 2 hours of incubation. During this incubation, endogenous Lipocalin-2 in the sample bound to the antibodies fixed on the inner surface of the wells. Non-reactive sample components were removed by a washing step. Afterwards, a biotinylated detection antibody was added. During a 2 hours incubation, a sandwich complex consisting of the two antibodies and the Lipocalin-2 was formed. Excess of detection antibody was washed out. Then, streptavidin conjugated to horseradish-peroxidase was added to complete the sandwich for 20 minutes of incubation. Excess of enzyme conjugate was washed out. Finally, a chromogenic substrate, TMB (3,3′,5,5′-Tetra-Methyl-Benzidine) was added to all wells. During 20 minutes of incubation, the substrate was converted to a colored end product (blue) by the fixed enzyme. Enzyme reaction was stopped by dispensing of hydrochloric acid as stop solution (change from blue to yellow). The color intensity was directly proportional to the concentration of Lipocalin-2 present in the sample. The optical density of the color solution was measured with a microplate reader at 450 nm.

TIMP-1: TIMP-1 level was determined by R&D Systems Luminex assays (R&D Systems, Bio-Techne, Lille, France). Measurement process were performed using the manufacturer instructions and the absorbance was read on a FLUOstar Omega (BMG labtech, Champigny-sur-Marne, France).

Results

Alpha-tropomyosin was identified as a 37-kDa antigen targeted by antibodies found in sera of patients with systemic autoimmune disease. Induction of pathogenic autoimmunity to α-tropomyosin was tested and confirmed in Lewis rats immunized with bovine α-tropomyosin in Complete Freund's Adjuvant (CFA). The immunized rats developed inflammatory lesions in the uveal tracts, joints and skin (Mor et al 2002 Eur. J. Immunol. 32:356-365). Thus, α-tropomyosin induces multiple symptoms and immune dysregulation closely correlated with those observed in patients with systemic inflammatory autoimmune diseases. The α-tropomyosin model was therefore used by the inventors as a model of vasculitis and of Behçet's disease.

In the experimental model, inflammation was observed as early as 14 days after the induction of the pathology in the eyes, joints and on the skin. It increased until it become widespread 21 days after induction (data not shown). The goal was to investigate the mechanisms involved by measuring inflammatory and regulatory mediators in the inflamed tissues of treated animals. Nitrite and urea concentration were assessed since Touri et al., 2018 showed that nitrite upregulation is associated with M1 macrophages activity and inflammation, while urea upregulation is associated with M2 macrophages activity and inflammation decrease.

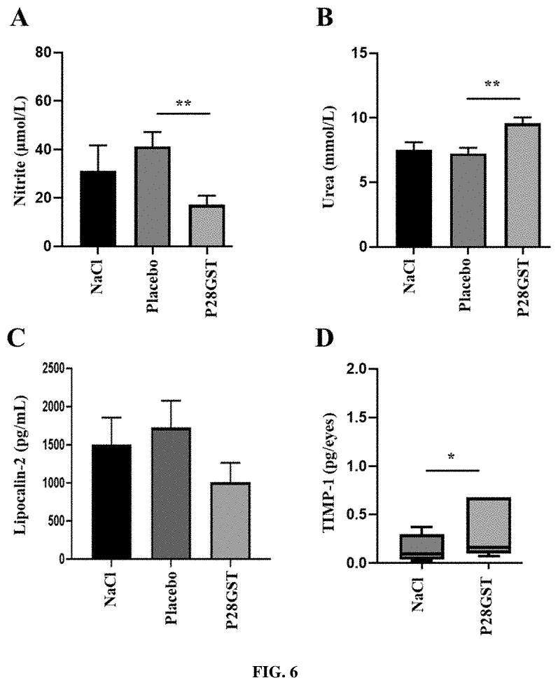

Twenty-five days after disease induction and thus 7 days after treatment initiation, plasmatic nitrite concentration significantly differed between groups. In particular, there was a significant decrease (p=0.007) of nitrite in the P28GST group compared to placebo (FIG. 6A). Thirty-two days after disease induction and thus 14 days after treatment initiation, plasmatic urea concentration significantly differed between groups. A significant increase (p=0.0045) was observed in the P28GST group compared to placebo (FIG. 6B). These results both show evidences of a resolution of the systemic inflammation.

Lipocalin-2 (LCN2 aka NGAL) recently emerged as a useful biomarker of inflammatory-mediated autoimmune diseases. The plasmatic concentration of Lipocalin-2 was then assessed. First, a significant increase of Lipocalin-2 serum concentration was observed between D0 and D18 (treatment initiation) confirming the onset of inflammation in all groups (data not shown). However, a slight decline of LCN2 serum concentration appeared in the P28GST treated group at 25 days (FIG. 6C).

This decline in the LCN2 serum concentration of rats treated with P28GST suggests a weakening of the disease.

Tissue inhibitor of metalloproteinase-1 (TIMP-1) is involved in the control of inflammation in many inflammatory-mediated autoimmune diseases and in areas of critical function (i.e., eyes). Its upregulation decreases inflammation and then organ destruction. Thirty-two days after disease induction, corresponding to the end of the study and to 14 days after treatment initiation, protein extracts from eyes showed a significant increase in TIMP-1 in the P28GST treated group compared to the control group (NaCl) (FIG. 6D). This significant difference (* p=0.0490) indicates that P28GST induced a regulation of inflammation.

Altogether, these results strongly suggested a positive effect of P28GST in systemic inflammatory disease.

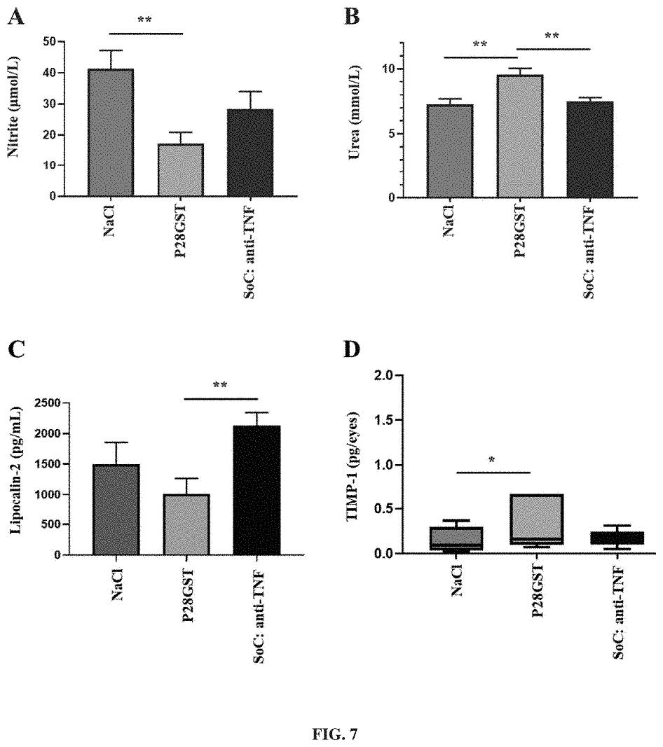

Finally, the P28GST effects were compared to the effects mediated by a standard of care which is the anti-TNFα treatment. Twenty-five days after disease induction and 7 days after treatment initiation, plasmatic nitrite concentration significantly differed between groups. There was a significant decrease in the P28GST treated group compared to control group (NaCl) (p=0.007). There was no significant difference between the anti-TNFα treated group and either the control group or the P28GST treated group (FIG. 7A). Thirty-two days after disease induction and 14 days after treatment initiation, plasmatic urea concentration significantly differed between groups. In particular, there was a significant increase (p=0.0045) in the P28GST treated group compared to control group (NaCl) and to the anti-TNFα treated groups (p=0.0067) (FIG. 7B). A significant increase of Lipocalin-2 serum concentration between D0 and D18 after disease induction confirms the onset of inflammation in all groups (data not shown). Twenty-five days after disease induction and 7 days after treatment initiation, a significant decline of LCN2 serum concentration only appeared in the P28GST treated group (p=0.0047) and not in the anti-TNFα treated group (FIG. 7C). Thirty-two days after disease induction and 14 days after treatment initiation, protein extracts from eyes showed a significant increase in TIMP-1 between the P28GST treated group and the control group (NaCl). This significant difference (p=0.049) indicates that P28GST induced a regulation of the inflammation, while anti-TNFα showed no such effect (FIG. 7D).

In conclusion, the results showed that P28GST may be better than anti-TNFα in treating systemic inflammatory diseases. Indeed, the anti-TNFα treatment induced no modification of the plasmatic urea concentrations, of the Timp-1 concentrations and of serum Lipocalin-2 concentrations, whereas the P28GST treatment did. The P28GST treatment thus induced a resolution of the systemic inflammation and weakened the symptoms of the disease.

Altogether, the results show that P28GST proteins from Schistosoma are capable of inducing M2-type immune response and/or reducing the M1-type immune response, i.e. of inducing a polarization towards a M2-type immune response. Indeed, the inventors showed that the P28GST proteins decreased the secretion of pro-inflammatory cytokines and mediators known to be produced by M1 macrophages, and increased the secretion of anti-inflammatory cytokines and mediators known to be produced by M2 macrophages. Moreover, this decrease in the M1-type response was associated to a reduction of the symptoms associated with inflammation.

In conclusion, P28GST proteins from Schistosoma can modulate the inflammatory response and represent a new approach for preventing or treating patients suffering from a disease characterized by a M1/M2 macrophage ratio dysregulation or patients with vasculitis.

1-15. (canceled)

16. A method of decreasing the M1-type immune response and/or increasing the M2-type immune response, in a subject in need thereof, comprising administering to said subject a therapeutically effective amount of a polypeptide comprising, or consisting of, an amino acid sequence selected from the group consisting of:
a) the sequence of SEQ ID NO: 1, SEQ ID NO: 2, SEQ ID NO: 3, SEQ ID NO: 5 SEQ ID NO: 6, SEQ ID NO: 7 or SEQ ID NO: 8;
b) a fragment of a sequence defined in a), provided that said polypeptide decreases the M1-type immune response and/or increases the M2-type immune response; and
c) a sequence having at least 80% of identity with a sequence defined in a) or b), provided that said polypeptide decreases the M1-type immune response and/or increases the M2-type immune response.

17. The method according to claim 16, for the preventive or therapeutic treatment of:
vasculitis, or
a disease characterized by a M1/M2 macrophage ratio dysregulation selected from the group consisting of atherosclerosis, endometriosis, hypertension, osteonecrosis, Parkinson's disease, steatohepatitis, obesity-induced pathologies, lipodystrophy and myocardial infarction.

18. A method of preventing or treating vasculitis, in a subject in need thereof, comprising administering to said subject a therapeutically effective amount of
a polypeptide comprising, or consisting of, an amino acid sequence selected from the group consisting of:
a) the sequence of SEQ ID NO: 1, SEQ ID NO: 2, SEQ ID NO: 3, SEQ ID NO: 5 SEQ ID NO: 6, SEQ ID NO: 7 or SEQ ID NO: 8;
b) a fragment of a sequence defined in a), provided that said polypeptide decreases the M1-type immune response and/or increases the M2-type immune response; and
c) a sequence having at least 80% of identity with a sequence defined in a) or b), provided that said polypeptide decreases the M1-type immune response and/or increases the M2-type immune response.

19. The method according to claim 18, wherein said polypeptide decreases the M1-type immune response and/or increases the M2-type immune response.

20. The method according to claim 16, wherein said fragment has an amino acid sequence selected from the group consisting of SEQ ID NO: 19 to SEQ ID NO: 51.

21. The method according to claim 16, wherein said polypeptide comprises, or consists of, an amino acid sequence selected from the group consisting of:
a) the sequence of SEQ ID NO: 1;
b) a fragment having an amino acid sequence selected from the group consisting of SEQ ID NO: 19 to SEQ ID NO: 30; and
c) a sequence having at least 80% of identity with a sequence defined in a) or b), provided that said polypeptide decreases the M1-type immune response and/or increases the M2-type immune response.

22. A method of preventing or treating vasculitis, or a disease characterized by a M1/M2 macrophage ratio dysregulation selected from the group consisting of atherosclerosis, endometriosis, hypertension, osteonecrosis, Parkinson's disease, steatohepatitis, obesity-induced pathologies, lipodystrophy and myocardial infarction, in a subject in need thereof, comprising administering to said subject a therapeutically effective amount of
a nucleic acid encoding a polypeptide comprising, or consisting of, an amino acid sequence selected from the group consisting of:
a) the sequence of SEQ ID NO: 1, SEQ ID NO: 2, SEQ ID NO: 3, SEQ ID NO: 5 SEQ ID NO: 6, SEQ ID NO: 7 or SEQ ID NO: 8;
b) a fragment of a sequence defined in a), provided that said polypeptide decreases the M1-type immune response and/or increases the M2-type immune response; and
c) a sequence having at least 80% of identity with a sequence defined in a) or b), provided that said polypeptide decreases the M1-type immune response and/or increases the M2-type immune response; or
a vector comprising said nucleic acid.

23. The method according to claim 16, wherein said polypeptide is administered in simultaneous, separate or sequential combination with at least one adjuvant.

24. The method according to claim 23, wherein said adjuvant is a natural or non-natural aluminum salt.

25. The method according to claim 16, wherein said subject suffers from vasculitis, atherosclerosis, endometriosis, hypertension, osteonecrosis, Parkinson's disease, steatohepatitis, obesity-induced pathologies, lipodystrophy, or myocardial infarction.

26. The method according to claim 25, wherein said vasculitis is selected from the group consisting of Behçet's disease (BD), Cogan's syndrome (CS), Takayasu arteritis (TAK), Giant cell arteritis (GCA), Polyarteritis nodosa (PAN), Kawasaki disease (KD), Antineutrophil cytoplasmic antibody (ANCA)-associated vasculitis (AAV), Microscopic polyangiitis (MPA), Granulomatosis with polyangiitis (Wegener's) (GPA), Eosinophilic granulomatosis with polyangiitis (Churg-Strauss) (EGPA)), Immune complex small vessel vasculitis, Anti-glomerular basement membrane (anti-GBM) disease, Cryoglobulinemic vasculitis (CV), IgA vasculitis (Henoch-Schanlein) (IgAV), Hypocomplementemic urticarial vasculitis (HUV) (anti-C1q vasculitis), Cutaneous leukocytoclastic angiitis, Cutaneous arteritis, Primary central nervous system vasculitis, Isolated aortitis.

27. The method according to claim 25, wherein said vasculitis is associated to another disease selected from the group consisting of Lupus, Rheumatoid arthritis, Sarcoidosis, Hepatitis C, Hepatitis B, Syphilis and Cancer.

28. The method according to claim 16, wherein said polypeptide is comprised in a pharmaceutical composition further comprising a pharmaceutically acceptable excipient, or in a vaccine composition further comprising at least one adjuvant.

29. The method according to claim 18, wherein said polypeptide is comprised in a pharmaceutical composition further comprising a pharmaceutically acceptable excipient, or in a vaccine composition further comprising at least one adjuvant.

30. The method according to claim 18, wherein said fragment has an amino acid sequence selected from the group consisting of SEQ ID NO: 19 to SEQ ID NO: 51.

31. The method according to claim 18, wherein said polypeptide comprises, or consists of, an amino acid sequence selected from the group consisting of:
a) the sequence of SEQ ID NO: 1;
b) a fragment having an amino acid sequence selected from the group consisting of SEQ ID NO: 19 to SEQ ID NO: 30; and
c) a sequence having at least 80% of identity with a sequence defined in a) or b), provided that said polypeptide decreases the M1-type immune response and/or increases the M2-type immune response.

32. The method according to claim 18, wherein said polypeptide is administered in simultaneous, separate or sequential combination with at least one adjuvant.

33. The method according to claim 32, wherein said adjuvant is a natural or non-natural aluminum salt.

34. The method according to claim 18, wherein said vasculitis is selected from the group consisting of Behçet's disease (BD), Cogan's syndrome (CS), Takayasu arteritis (TAK), Giant cell arteritis (GCA), Polyarteritis nodosa (PAN), Kawasaki disease (KD), Antineutrophil cytoplasmic antibody (ANCA)-associated vasculitis (AAV), Microscopic polyangiitis (MPA), Granulomatosis with polyangiitis (Wegener's) (GPA), Eosinophilic granulomatosis with polyangiitis (Churg-Strauss) (EGPA)), Immune complex small vessel vasculitis, Anti-glomerular basement membrane (anti-GBM) disease, Cryoglobulinemic vasculitis (CV), IgA vasculitis (Henoch-Schanlein) (IgAV), Hypocomplementemic urticarial vasculitis (HUV) (anti-C1q vasculitis), Cutaneous leukocytoclastic angiitis, Cutaneous arteritis, Primary central nervous system vasculitis, Isolated aortitis.

35. The method according to claim 18, wherein said vasculitis is associated to another disease selected from the group consisting of Lupus, Rheumatoid arthritis, Sarcoidosis, Hepatitis C, Hepatitis B, Syphilis and Cancer.


SEQUENCE LISTING (51 sequences)

SEQ ID NO: 1 - protein, 211 aa, Schistosoma haematobium
  Sh28GST
MTGDHIKVIYFNGRGRAESIRMTLVAAGVNYEDERISFQDWPKIKPTIPGGRLPAVKITDNHGHVKWMVESLAIARYMAKKHHMMGGTEEEYYNVEKLIGQAEDLEHEYYKTLMKPEEEKQKIIKEILNGKVPVLLDIICESLKASTGKLAVGDKVTLADLVLIAVIDHVTDLDKEFLTGKYPEIHKHRENLLASSPRLAKYLSDRAATPF

SEQ ID NO: 2 - protein, 211 aa, Schistosoma mansoni
  Sm28GST
MAGEHIKVIYFDGRGRAESIRMTLVAAGVDYEDERISFQDWPKIKPTIPGGRLPAVKVTDDHGHVKWMLESLAIARYMAKKHHMMGETDEEYYSVEKLIGQAEDVEHEYHKTLMKPQEEKEKITKEILNGKVPVLLNMICESLKGSTGKLAVGDKVTLADLVLIAVIDHVTDLDKGFLTGKYPEIHKHRENLLASSPRLAKYLSNRPATPF

SEQ ID NO: 3 - protein, 211 aa, Schistosoma bovis
  Sb28GST
MTGDHIKVIYFNGRGRAESIRMTLVAAGVNYEDERISFQDWPKIKPTIPGGRLPAVKITDNHGHVKWMLESLAIARYMAKKHHMMGETDEEYYNVEKLIGQVEDLEHEYHKTLMKPEEEKQKITKEILNGKVPVLLDIICESLKASTGKLAVGDKVTLADLVLIAVIDHVTDLDKEFLTGKYPEIHKHRENLLASSPRLAKYLSDRAATPF

SEQ ID NO: 4 - DNA, 792 bp, Schistosoma haematobium
  Sh28GST
tctgtctgactgtatgatgactggtgatcatatcaaggttatctattttaacggacgcggacgagctgaatcgatccggatgacacttgtggcagctggtgtgaactacgaagatgagagaattagtttccaagattggccgaaaatcaaaccaactattccgggcggacgattgcctgcagtgaaaatcaccgataatcatgggcacgtgaaatggatggtagagagtttggctattgcacggtatatggcgaagaagcatcatatgatgggaggaacagaagaggagtattataatgttgagaagttgattggtcaggctgaagatctagaacatgaatattacaaaactttgatgaagccagaagaagagaaacagaagataatcaaagagatactgaacggcaaagtaccagttcttctcgatattatctgcgaatctctgaaagcgtccacaggcaagctggctgttggggataaagtgactctagccgacttagttctgattgctgtcattgaccatgtgactgatctggataaagaatttctaactggcaagtatcctgagatccataaacatagagaaaatctactagccagttcaccgagattggcgaaatatttatcagacagggctgcaactcccttctagaactgtcaacagaatgctgggtgtgacgagattgaagatactgatagtagtgcactggtgcgacctttttactaagacgtcatttgttttatggtattttttttcgcaatcgttattaaaataaacttagttttctgttt

SEQ ID NO: 5 - protein, 211 aa, Schistosoma japonicum
  Sj28GST
MACGHVKLIYFNGRGRAEPIRMILVAAGVEFEDERIEFQDWPKIKPTIPGGRLPIVKITDKRGDVKTMSESLAIARFIARKHNMMGDTDDEYYIIEKMIGQVEDVESEYHKTLMKPPEEKEKISKEILNGKVPILLQAICETLKESTGNLTVGDKVTLADVVLIASIDHITDLDKEFLTGKYPEIHKHRKHLLATSPKLAKYLSERHATAF

SEQ ID NO: 6 - protein, 218 aa, Schistosoma mansoni
  Sm26GST isoform 1
MAPKFGYWKVKGLVQPTRLLLEHLEETYEERAYDRNEIDAWSNDKFKLGLEFPNLPYYIDGDFKLTQSMAIIRYIADKHNMLGACPKERAEISMLEGAVLDIRMGVLRIAYNKEYETLKVDFLNKLPGRLKMFEDRLSNKTYLNGNCVTHPDFMLYDALDVVLYMDSQCLNEFPKLVSFKKCIEDLPQIKNYLNSSRYIKWPLQGWDATFGGGDTPPK

SEQ ID NO: 7 - protein, 195 aa, Schistosoma mansoni
  Sm26GST isoform 2
MAPKLGYWKIKGLVQPTRLLLEYLGEAYEERLYDRNDGDVWRNEKFKLGLDFPNLPYYIDGDVKLTQSMAILRYIADKHNMLGGCPKERAEISMLEGAILDIRYGVSRIAYNKEFETLKVDFLNQLPGMLKMFEDRLSHNTYLNGDKVTHPDFMLYDALDVNLPPIKNYLNSNRYIKWPLQGWSATFGGGDAPPK

SEQ ID NO: 8 - protein, 218 aa, Schistosoma japonicum
  Sj26GST
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 9 - DNA, 20 bp, Artificial Sequence
  IL-1b forward primer
agctctccacctcaatggac

SEQ ID NO: 10 - DNA, 20 bp, Artificial Sequence
  IL-1b reverse primer
aggccacaggtattttgtcg

SEQ ID NO: 11 - DNA, 19 bp, Artificial Sequence
  TNFa forward primer
cctgtagcccacgtcgtag

SEQ ID NO: 12 - DNA, 22 bp, Artificial Sequence
  TNFa reverse primer
gggagtagacaaggtacaaccc

SEQ ID NO: 13 - DNA, 21 bp, Artificial Sequence
  b-actin forward primer
ccttcttgggtatggaatcct

SEQ ID NO: 14 - DNA, 21 bp, Artificial Sequence
  b-actin reverse primer
ctttacggatgtcaacgtcac

SEQ ID NO: 15 - DNA, 21 bp, Artificial Sequence
  iNOS forward primer
cagctgggctgtacaaacctt

SEQ ID NO: 16 - DNA, 21 bp, Artificial Sequence
  iNOS reverse primer
cattggaagtgaagcgtttca

SEQ ID NO: 17 - DNA, 20 bp, Artificial Sequence
  Arg1 forward primer
cagaagaatggaagagtcag

SEQ ID NO: 18 - DNA, 20 bp, Artificial Sequence
  Arg1 reverse primer
cagatatgcagggagtcacc

SEQ ID NO: 19 - protein, 20 aa, Schistosoma haematobium
LVAAGVNYEDERISFQDWPK

SEQ ID NO: 20 - protein, 20 aa, Schistosoma mansoni
LVAAGVDYEDERISFQDWPK

SEQ ID NO: 21 - protein, 20 aa, Schistosoma bovis
LVAAGVNYEDERISFQDWPK

SEQ ID NO: 22 - protein, 20 aa, Schistosoma japonicum
LVAAGVEFEDERIEFQDWPK

SEQ ID NO: 23 - protein, 17 aa, Schistosoma haematobium
KPEEEKQKIIKEILNGK

SEQ ID NO: 24 - protein, 17 aa, Schistosoma mansoni
KPQEEKEKITKEILNGK

SEQ ID NO: 25 - protein, 17 aa, Schistosoma bovis
KPEEEKQKITKEILNGK

SEQ ID NO: 26 - protein, 17 aa, Schistosoma japonicum
KPPEEKEKISKEILNGK

SEQ ID NO: 27 - protein, 22 aa, Schistosoma haematobium
ENLLASSPRLAKYLSDRAATPF

SEQ ID NO: 28 - protein, 22 aa, Schistosoma mansoni
ENLLASSPRLAKYLSNRPATPF

SEQ ID NO: 29 - protein, 22 aa, Schistosoma bovis
ENLLASSPRLAKYLSDRAATPF

SEQ ID NO: 30 - protein, 22 aa, Schistosoma japonicum
KHLLATSPKLAKYLSERHATAF

SEQ ID NO: 31 - protein, 46 aa, Schistosoma haematobium
GRAESIRMTLVAAGVNYEDERISFQDWPKIKPTIPGGRLPAVKITD

SEQ ID NO: 32 - protein, 46 aa, Schistosoma mansoni
GRAESIRMTLVAAGVDYEDERISFQDWPKIKPTIPGGRLPAVKVTD

SEQ ID NO: 33 - protein, 46 aa, Schistosoma bovis; identical to SEQ ID NO: 31

SEQ ID NO: 34 - protein, 46 aa, Schistosoma japonicum
GRAEPIRMILVAAGVEFEDERIEFQDWPKIKPTIPGGRLPIVKITD

SEQ ID NO: 35 - protein, 51 aa, Schistosoma haematobium
GQAEDLEHEYYKTLMKPEEEKQKIIKEILNGKVPVLLDIICESLKASTGKL

SEQ ID NO: 36 - protein, 51 aa, Schistosoma mansoni
GQAEDVEHEYHKTLMKPQEEKEKITKEILNGKVPVLLNMICESLKGSTGKL

SEQ ID NO: 37 - protein, 51 aa, Schistosoma bovis
GQVEDLEHEYHKTLMKPEEEKQKITKEILNGKVPVLLDIICESLKASTGKL

SEQ ID NO: 38 - protein, 51 aa, Schistosoma japonicum
GQVEDVESEYHKTLMKPPEEKEKISKEILNGKVPILLQAICETLKESTGNL

SEQ ID NO: 39 - protein, 42 aa, Schistosoma haematobium
VTDLDKEFLTGKYPEIHKHRENLLASSPRLAKYLSDRAATPF

SEQ ID NO: 40 - protein, 42 aa, Schistosoma mansoni
VTDLDKGFLTGKYPEIHKHRENLLASSPRLAKYLSNRPATPF

SEQ ID NO: 41 - protein, 42 aa, Schistosoma bovis; identical to SEQ ID NO: 39

SEQ ID NO: 42 - protein, 42 aa, Schistosoma japonicum
ITDLDKEFLTGKYPEIHKHRKHLLATSPKLAKYLSERHATAF

SEQ ID NO: 43 - protein, 23 aa, Schistosoma mansoni
LEHLEETYEERAYDRNEIDAWSN

SEQ ID NO: 44 - protein, 23 aa, Schistosoma mansoni
LEYLGEAYEERLYDRNDGDVWRN

SEQ ID NO: 45 - protein, 23 aa, Schistosoma japonicum
LEYLEEKYEEHLYERDEGDKWRN

SEQ ID NO: 46 - protein, 14 aa, Schistosoma mansoni
NKEYETLKVDFLNK

SEQ ID NO: 47 - protein, 14 aa, Schistosoma mansoni
NKEFETLKVDFLNQ

SEQ ID NO: 48 - protein, 14 aa, Schistosoma japonicum
SKDFETLKVDFLSK

SEQ ID NO: 49 - protein, 37 aa, Schistosoma mansoni
KCIEDLPQIKNYLNSSRYIKWPLQGWDATFGGGDTPP

SEQ ID NO: 50 - protein, 16 aa, Schistosoma mansoni
SNRYIKWPLQGWSATF

SEQ ID NO: 51 - protein, 23 aa, Schistosoma japonicum
KRIEAIPQIDKYLKSSKYIAWPL